(19) United States
(12) Patent Application Publication
(10) Pub. No.: US 2011/0287964 A1
(43) Pub. Date: Nov. 24, 2011

(54) URINARY BIOMARKERS FOR SENSITIVE AND SPECIFIC DETECTION OF ACUTE KIDNEY INJURY IN HUMANS

(75) Inventors: Joseph V. Bonventre, Wayland, MA (US); Vishal S. Vaidya, Cambridge, MA (US)

(73) Assignee: THE BRIGHAM AND WOMEN'S HOSPITAL, INC., Boston, MA (US)

(21) Appl. No.: 13/129,600

(22) PCT Filed: Nov. 17, 2009
(86) PCT No.: PCT/US2009/064795

§ 371 Date: Aug. 8, 2011

(51) Int. Cl. C40B 30/04; G01N 33/573; C12Q 1/34; G01N 33/566

(52) U.S. Cl. 506/9; 436/501; 435/7.4; 435/18

(57) ABSTRACT

The present invention is directed to acute kidney injury biomarkers, and methods and kits comprising the use of agents directed against acute kidney injury biomarkers for facilitating and enhancing the diagnosis of AKI. The present invention is based on the discovery that specific biomarkers are present in urine at higher concentrations in subjects with acute kidney injury (AKI) as compared with subjects that have no symptoms of AKI. The invention is directed to methods for diagnosis of AKI by determining and monitoring the levels of at least one biomarker protein in a biological sample, such as urine. Further, the invention is directed to methods for facilitating the distinction of kidney infection from bladder infection in a subject.

### CROSS REFERENCE TO RELATED APPLICATIONS

This application claims the benefit under 35 U.S.C. §119(e) of U.S. Provisional Patent Application Ser. No. 61/115,242 filed on Nov. 17, 2008, the contents of which are incorporated herein in their entirety by reference.

### GOVERNMENT SUPPORT

This work was made with Government support under Grant No. DK074099, awarded by the National Institutes of Health. The Government has certain rights to the invention.

### FIELD OF THE INVENTION

The present invention relates generally to the use of urinary biomarkers for sensitive and specific detection of acute kidney injury as well as for distinguishing kidney infection from bladder infection in humans by assessing the levels of biomarkers in urine.

### BACKGROUND OF THE INVENTION

Acute kidney injury (AKI) is associated with high morbidity and mortality: the mortality rate in hospital intensive care units ranges from 40% to 80%. The lack of sensitive and specific injury biomarkers greatly impedes the development of therapeutic strategies to improve outcomes of AKI. The traditional blood (creatinine, blood urea nitrogen) and urine markers of kidney injury (casts, fractional excretion of sodium, urinary concentrating ability), that have been used for decades in clinical studies for diagnosis and prognosis of AKI, are insensitive, nonspecific, and do not directly reflect injury to kidney cells. Outside of the clinical setting, the lack of AKI biomarkers has impeded the development of drugs and therapies that may improve the devastating outcomes of AKI. Hence, there remains an urgent need for easily quantifiable and sensitive biomarkers for detecting and monitoring AKI.

Urinary tract infections (UTI) are relatively common: over 60% of women experience at least one UTI. Most UTI cases are known as cystitis, which involves the lower urinary tract (the bladder and urethra), and are painful such that treatment is sought before the bladder is damaged or the infection spreads. A UTI may spread to the upper tract (the ureters and kidneys), however, causing pyelonephritis (kidney infection), which can cause permanent kidney damage or even death. For example, the mortality rate exceeds 40% in kidney infection that obstructs the ureter. Perhaps half of all women experiencing a lower UTI may have an upper UTI as well. In the U.S., about 250,000 women per year develop pyelonephritis, and 100,000 are hospitalized for treatment. Hence, because of the risks involved, and because cystitis and pyelonephritis often require different therapeutical interventions, there is a need for a biomarker useful in distinguishing between cystitis and pyelonephritis.

### SUMMARY OF THE INVENTION

Provided herein are biomarkers, and methods, assays and kits comprising such biomarkers, that are useful in diagnosing and monitoring acute kidney injury in patients. The present invention is based on the discovery that specific biomarkers are present in urine at higher concentrations in subjects with acute kidney injury (AKI) as compared with subjects that have no symptoms of AKI. Accordingly, the invention is directed to methods for diagnosis of AKI by determining and monitoring the levels of at least one biomarker protein in a biological sample, such as urine. Further, the invention is directed to methods for facilitating the distinction of kidney infection from bladder infection in a subject.

Accordingly, one aspect of the invention provides at least one biomarker specific for the diagnosis and monitoring of acute kidney injury in a subject in need thereof.

One embodiment of this aspect, and all aspects described herein, provides a single urinary biomarker, hepatocyte growth factor (HGF), that is significantly elevated in patients with AKI. Another embodiment of this aspect, and all aspects described herein, provides a urinary biomarker, kidney injury molecule-1 (KIM-1), as a biomarker for kidney infection in patients exhibiting symptoms of bladder infection.

Another embodiment of this aspect, and all aspects described herein, provides a panel of biomarkers, each of which is elevated in AKI patients, and provides comparative value in the diagnosis and prognosis of AKI. In one embodiment of this aspect, and all aspects described herein, the AKI biomarker panel comprises kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10). In another embodiment of this aspect, and all aspects described herein, the AKI biomarker panel consists essentially of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10). In another embodiment of this aspect, and all aspects described herein, the AKI biomarker panel consists of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10).

In another embodiment of this aspect, and all aspects described herein, the AKI biomarker panel comprises KIM-1, NAG, HGF, and VEGF. In another embodiment of this aspect, and all aspects described herein, the AKI biomarker panel consists essentially of KIM-1, NAG, HGF, and VEGF. In another embodiment of this aspect, and all aspects described herein, the AKI biomarker panel consists of KIM-1, NAG, HGF, and VEGF.

In yet another embodiment of this aspect, and all aspects described herein, the AKI biomarker panel comprises KIM-1, NAG, and HGF. In another embodiment of this aspect, and all aspects described herein, the AKI biomarker panel consists essentially of KIM-1, NAG, and HGF. In another embodiment of this aspect, and all aspects described herein, the AKI biomarker panel consists of KIM-1, NAG, and HGF.

In all such embodiments of this aspect and all aspects described herein, an agent specific for total protein or a normalizing protein, such as creatinine, may also be included, or an assay to measure the level or concentration of total protein or a normalizing protein may be performed in order to provide a level or concentration to which the panel of biomarkers can be normalized to, in order to permit various comparisons, for example, between subject samples, or between a series of samples isolated from one subject at different timepoints.

In one embodiment of the invention, AKI biomarker levels (e.g., HGF) present in a biological sample, such as urine, are measured by contacting the test sample, or preparation thereof, with an agent, such as an antibody-based agent, that specifically binds to at least one AKI biomarker, or to a portion thereof, wherein the agent forms a complex with the biomarker which can be used in assays to determine the biomarker concentration or level. Any means known to those skilled in art can be used to assess biomarker levels. For example, biomarker levels can be assessed by ELISA, multiplex bead assay, or mass spectrometry, including SELDI mass spectrometry.

In another aspect, the invention provides methods of optimizing therapeutic efficacy for treatment of acute kidney injury.

Accordingly, in one embodiment of this aspect, the method comprises (a) measuring a level or concentration of at least one biomarker in a panel of biomarkers comprising a kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10); and (b) comparing the level or concentration of the at least one biomarker with a reference level or concentration of the at least one biomarker, wherein an increase in the level or concentration of at least one biomarker in a panel of biomarkers comprising KIM-1, NGAL, IL-18, HGF, Cys, NAG, VEGF, and CXCL10 in the sample relative to the reference level or concentration of said at least one biomarker indicates a need to administer to the subject a therapeutic treatment for acute kidney injury. In some embodiments, the biological sample is a urine sample.

In another embodiment of this aspect, the method comprises contacting a biological sample obtained from a subject with at least one agent specific for at least one biomarker in a panel of biomarkers comprising a kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10); (b) measuring a level or concentration of the at least one biomarker using an assay specific for the at least one agent; and (c) comparing the level or concentration of the at least one biomarker with a reference level or concentration of the at least one biomarker, wherein an increase in the level or concentration of at least one biomarker in a panel of biomarkers comprising KIM-1, NGAL, IL-18, HGF, Cys, NAG, VEGF, and CXCL10 in the sample relative to the reference level or concentration of said at least one biomarker indicates a need to administer to the subject a therapeutic treatment for acute kidney injury. In some embodiments, the biological sample is a urine sample.

In another aspect, the invention provides for kits that comprise means for measuring at least one AKI biomarker, for example, HGF, in a biological sample. The kit comprises a container for holding a biological sample (e.g. urine sample), and at least one agent, such as an antibody, that specifically binds at least one AKI biomarker for use in determining the level, concentration or the presence of at least one AKI biomarker in a biological sample, such as a urine sample.

In one embodiment of this aspect, the kit comprises at least one antibody that specifically binds to at least one AKI biomarker and an antibody for immobilization. In one such embodiment, one antibody is immobilized on a solid phase and the at least one antibody specific for at least one biomarker is detectably labeled. The kits can comprise anti-HGF, anti-KIM-1, anti-NGAL, anti-IL-18, anti-Cys, anti-NAG, anti-VEGF, or anti-IP-10 antibodies.

Another aspect described herein relates to a computer readable storage medium having computer readable instructions recorded thereon to define software modules for implementing on a computer a method for diagnosing acute kidney injury of at least one individual, the computer readable storage medium comprising: (a) instructions for storing and accessing data representing a level of at least one biomarker and a level of a normalizing protein determined for a biological sample obtained from at least one individual; (b) instructions for normalizing the level of the at least one biomarker to the level of normalizing protein via a normalization module, thereby producing a normalized level of the at least one biomarker, (c) instructions for comparing the normalized level of the at least one biomarker to reference data stored on the storage device using a comparison module, wherein the comparing step produces a retrieved content, and (d) instructions for displaying a page of the retrieved content for the user, wherein the retrieved content displays if there is a change in the normalized level of the at least one biomarker, thereby determining whether the at least one individual has acute kidney injury. In one embodiment, the normalizing protein is creatinine. In one embodiment, the normalizing protein is total protein. In one embodiment, the biological sample is a urine sample.

Also described herein is a computer system for obtaining data from a biological sample obtained from at least one individual, the system comprising: (a) a specimen container to hold a biological sample; (b) a determination module configured to determine reporter molecule information, wherein the reporter molecule information comprises 1) information representing binding of an agent to a normalizing protein, and 2) information representing binding of an agent to at least one biomarker; (c) a storage device configured to store data output from the determination module; (d) a normalization module configured to normalize reporter molecule information representing binding of an agent to at least one biomarker to reporter molecule information representing binding of an agent to normalizing protein; (e) a comparison module adapted to compare the data obtained from the normalization module with reference data on the storage device, wherein the comparison module produces a retrieved content; and (f) a display module for displaying a page of the retrieved content for the user, wherein the retrieved content displays if there is a change in the normalized level of the at least one biomarker, thereby determining whether the at least one individual has acute kidney injury. In one embodiment, the normalizing protein is creatinine. In one embodiment, the normalizing protein is total protein. In one embodiment, the biological sample is a urine sample.

### DEFINITIONS

As used herein, “acute kidney injury”, also known as “acute renal failure (ARF)” or “acute kidney failure”, refers to a disease or condition where a rapid loss of renal function occurs due to damage to the kidneys, resulting in retention of nitrogenous (urea and creatinine) and non-nitrogenous waste products that are normally excreted by the kidney. Depending on the severity and duration of the renal dysfunction, this accumulation is accompanied by metabolic disturbances, such as metabolic acidosis (acidification of the blood) and hyperkalaemia (elevated potassium levels), changes in body fluid balance, and effects on many other organ systems. It can be characterized by oliguria or anuria (decrease or cessation of urine production), although nonoliguric ARF may occur. Acute kidney injury may be a consequence of various causes including a) pre-renal (causes in the blood supply), which includes, but is not limited to, hypovolemia or decreased blood volume, usually from shock or dehydration and fluid loss or excessive diuretics use; hepatorenal syndrome, in which renal perfusion is compromised in liver failure; vascular problems, such as atheroembolic disease and renal vein thrombosis, which can occur as a complication of nephrotic syndrome; infection, usually sepsis, and systemic inflammation due to infection; severe burns; sequestration due to pericarditis and pancreatitis; and hypotension due to antihypertensives and vasodilators; b) intrinsic renal damage, which includes, but is not limited to, toxins or medication (e.g. some NSAIDs, aminoglycoside antibiotics, iodinated contrast, lithium, phosphate nephropathy due to bowel preparation for colonoscopy with sodium phosphates); rhabdomyolysis or breakdown of muscle tissue, where the resultant release of myoglobin in the blood affects the kidney, which can also be caused by injury (especially crush injury and extensive blunt trauma), statins, stimulants and some other drugs; hemolysis or breakdown of red blood cells, which can be caused by various conditions such as sickle-cell disease, and lupus erythematosus; multiple myeloma, either due to hypercalcemia or “cast nephropathy”; acute glomerulonephritis which may be due to a variety of causes, such as anti glomerular basement membrane disease/Goodpasture's syndrome, Wegener's granulomatosis or acute lupus nephritis with systemic lupus erythematosus; and c) post-renal causes (obstructive causes in the urinary tract) which include, but are not limited to, medication interfering with normal bladder emptying (e.g. anticholinergics); benign prostatic hypertrophy or prostate cancer; kidney stones; abdominal malignancy (e.g. ovarian cancer, colorectal cancer); obstructed urinary catheter; or drugs that can cause crystalluria and drugs that can lead to myoglobinuria & cystitis.

As used herein, a “subject” refers to a mammal, preferably a human. The term “individual”, “subject”, and “patient” are used interchangeably herein, and refer to an animal, for example a mammal, such as a human. The term “mammal” is intended to encompass a singular “mammal” and plural “mammals,” and includes, but is not limited: to humans, non-human primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and bears.

As used herein, the terms “sample” or “biological sample” refers to a sample of biological fluid, tissue, or cells, in a healthy and/or pathological state obtained from a subject. Such samples include, but are not limited to, urine, whole blood, serum, plasma, sputum, saliva, amniotic fluid, lymph fluid, tissue or fine needle biopsy samples, peritoneal fluid, cerebrospinal fluid, nipple aspirates, and includes supernatant from cell lysates, lysed cells, cellular extracts, and nuclear extracts. In some embodiments, the whole blood sample is further processed into serum or plasma samples. In some embodiments, a sample is taken from a human subject, and in alternative embodiments the sample is taken from any mammal, such as rodents, animal models of diseases, commercial animals, companion animals, dogs, cats, sheep, cattle, and pigs, etc. The sample can be pretreated as necessary for storage or preservation, by dilution in an appropriate buffer solution or concentrated, if desired. Any of a number of standard aqueous buffer solutions, employing one of a variety of buffers, such as phosphate, Tris, or the like, at physiological pH can be used. The sample can in certain circumstances be stored for use prior to use in the assays as disclosed herein. Such storage can be at +4° C. or frozen, for example at −20° C. or −80° C.

As used herein, the term “biomarker” or “urinary biomarker” refers to a polypeptide expressed endogenously in an individual or found or sequestered in a sample from an individual. The term “acute kidney injury biomarker” is used throughout the specification as an example of a type of biomarker useful with the methods described herein. Acute kidney injury and pyelonephritis are examples of conditions associated with a biomarker as the term “biomarker” is used herein. A urinary biomarker or acute kidney injury biomarker can include at least one of hepatocyte growth factor (HGF), kidney injury molecule-1 (KIM-1), neutrophil gelatinase associated lipocalin (NGAL), interleukin-18 (IL-18), cystatin C (Cys), N-acetyl-β-D-glucosaminidase (NAG), vascular endothelial growth factor (VEGF), and chemokine interferon-inducible protein 10 (IP-10; CXCL10). For each of the biomarkers useful for diagnosing AKI, e.g., KIM-1, NGAL, VEGF, Cys, CXCL10, IL-18, NAG, and HGF, a reference to the specific protein also encompasses domains or fragments of those proteins, as well as species, variants, homologues, allelic forms, mutant forms, and equivalents thereof.

As used herein the term “agent” refers to a protein-binding agent that permits detection and/or quantification of levels, concentrations, expression levels, or activity of the total protein in a biological sample, a normalizing protein (e.g., actin), or an acute kidney injury biomarker in a sample. Such agents include, but are not limited to, antibodies, recombinant antibodies, chimeric antibodies, tribodies, midibodies, protein-binding agents, small molecules, recombinant protein, peptides, aptamers, avimers and protein-binding derivatives or fragments thereof. As used herein, the phrase “agent specific for at least one biomarker” refers to a protein-binding agent that permits detection and/or quantification of levels, concentrations, or expression levels for a biomarker. Such agents include, but are not limited to, antibodies, recombinant antibodies, chimeric antibodies, tribodies, midibodies, protein-binding agents, small molecules, recombinant protein, peptides, aptamers, avimers and protein-binding derivatives or fragments thereof. As defined herein, an agent upon binding a specific biomarker, normalizing protein, or total protein forms an “agent-biomarker complex,” “agent-normalizing protein complex,” or “agent-total protein complex.” As used herein, the term “reporter molecule information” refers to data derived from a signal indicating binding of an agent to or complex formation with an acute kidney injury biomarker in a sample, i.e., formation of an agent-biomarker complex,” “agent-normalizing protein complex,” or “agent-total protein complex.” A signal can comprise e.g., light, fluorescence, colorimetric or other detectable signal that indicates agent binding to an acute kidney injury biomarker, a normalizing protein, or total protein

As used herein the term “comprising” or “comprises” is used in reference to compositions, methods, and respective component(s) thereof, that are essential to the invention, yet open to the inclusion of unspecified elements, whether essential or not. As used herein the term “consisting essentially of” refers to those elements required for a given embodiment. The term permits the presence of elements that do not materially affect the basic and novel or functional characteristic(s) of that embodiment of the invention. The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

### BRIEF DESCRIPTION OF THE DRAWINGS

The accompanying drawings, which are incorporated in and constitute a part of this specification, illustrate embodiments of the invention and, together with the description, serve to explain the objects, advantages, and principles of the invention.

FIGS. 1A-1F show the evaluation of microbead based assay for quantification of human urinary KIM-1, NGAL, IL-18, HGF, VEGF and IP-10. FIG. 1A shows the standard curve for human KIM-1 obtained using purified recombinant human KIM-1 ectodomain fusion protein. It demonstrated linearity over five orders of magnitude from 40 pg/ml to 160,000 pg/ml with the lowest limit of detection (LLD) to be 4.4 pg/ml. FIG. 1B shows the standard curve for human NGAL obtained using a commercially available purified NGAL protein. The NGAL standard curve was also linear over five orders of magnitude from 0.49 to 1000 ng/ml with the LLD of 534 pg/ml. The standard curves for IL-18 (FIG. 1C) and HGF (Figure D) ranged from 0.12 pg/ml to 2000 pg/ml and 0.7 pg/ml to 1446 pg/ml with the LLD of 125 fg/ml and 709 fg/ml respectively. Similarly, the standard curves for VEGF (Figure E) and IP-10 (Figure F) ranged from 7.8 pg/ml to 31982 pg/ml and 25 pg/ml to 10000 pg/ml with the LLD of 10 pg/ml and 32 pg/ml respectively. The standard curves were plotted as five parameter logistic curves and repeated eight times on different sets of samples on different days using different sets of beads coupled with different batches of primary antibody. The inset in each panel documents the linearity of the maximum fluorescence intensity at lower concentrations.

FIGS. 2A-2I show a scatterplot of human urinary KIM-1 (Figure A), Protein (Figure B), NGAL (Figure C), HGF (Figure D), IP-10 (Figure E), Cystatin C (Figure F), IL-18 (Figure G), NAG (Figure H) and VEGF (FIG. 1) in patients with and without acute kidney injury. Urinary biomarker measurements were normalized to urine creatinine and were plotted on a logarithmic Y-axis. The number of patients in each group is indicated below each category on the X-axis.

FIG. 3 depicts a schematic of the structure of Kim-1.

FIG. 4 shows a block diagram depicting an exemplary system for diagnosis of acute kidney injury.

FIG. 5 depicts an exemplary set of instructions on a computer readable storage medium for use with the systems described herein.

### DETAILED DESCRIPTION OF THE INVENTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Acute kidney injury (AKI) is associated with high morbidity and mortality. The lack of sensitive and specific injury biomarkers has greatly impeded the development of therapeutic strategies to improve outcomes of AKI. The diagnostic approach to AKI has stagnated and rests today upon the same “legacy” biomarkers—BUN, creatinine, and urine output—that do not directly reflect cell injury but rather delayed functional consequences of the injury. This has greatly impeded therapeutic innovation. A first step in the validation of novel biomarkers of AKI is the demonstration that established AKI can be distinguished from non-AKI controls.

The embodiments of the present invention provide for the diagnostic performance of nine urinary biomarkers of AKI—kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), chemokine interferon-inducible protein 10 (IP-10, CXCL 10), and total protein. These biomarkers were analyzed in a cross-sectional comparison of 204 patients with or without AKI.

Median urinary concentrations of each of these biomarkers was significantly higher in patients with AKI than in those without AKI (P<0.001). More specifically, the area under the receiver operating characteristics curve (AUC-ROC) for the combination of biomarkers using a logic regression model [risk score of 2.93*(NGAL>5.72 and HGF>0.17)+2.93*(PROTEIN>0.22)-2*(KIM<0.58)] was significantly greater (0.94) than individual biomarker AUC-ROC's. Age-adjusted levels of urinary KIM-1, NAG, HGF, VEGF and total protein were significantly higher in patients who died or required renal replacement therapy (RRT) when compared to those who survived and did not require RRT. These results demonstrate the comparative value of these biomarkers in the diagnosis and prognosis of AKI.

Several of the biomarkers provided for herein in the context of AKI have been characterized previously to some extent. KIM-1 (also known as TIM-1 or HAVCR-1), is a type I cell membrane glycoprotein, which is up-regulated about 50-fold to 100-fold in the kidney, and the ectodomain of KIM-1 is shed into the urine in both rodents (Ichimura et al., 273 J. Biol. Chem. 4135-42 (1998); Vaidya et al., 2 Expert Opin. Drug Metab. Toxicol. 697-713 (2006)), and humans (Han et al., 62 Kidney Int'l 237-44 (2002)), after proximal tubular kidney injury.

There have been studies suggesting that urinary NGAL levels increased 10-fold to 100-fold in rodents after cisplatin-induced nephrotoxicity, and in patients with ischemic and septic AKI (More et al., 115 J. Clin. Invest. 610-21 (2005)). Also, high levels of urinary NGAL predicted the onset of AKI two hours after cardiopulmonary bypass in children undergoing cardiac surgery, two to four days before AKI was identified by changes in serum creatinine (Mishra et al., 365 Lancet 1231-38 (2005)).

Urinary Cys-C levels have been found to be elevated in individuals with known tubular dysfunction (Conti et al., 44 Urinary Chem. Lab. Med. 288-91 (2006); Uchida & Gotoh 323 Clin. Chim Acta 121-28 (2002)). Others have reported that elevated urinary Cys-C levels were highly predictive of poor outcome (requirement for RRT) in a heterogeneous group of patients with initially nonoliguric AKI (Herget-Rosenthal et al., 50 Clin. Chem. 552-58.(2004)).

Urinary IL-18 levels are elevated in patients with AKI and delayed graft function compared with normal subjects. Elevation of urinary IL-18 could predict AKI one day before creatinine in 138 patients with adult respiratory distress syndrome (ARDS) and IL-18 levels were independent predictors of mortality at the time of mechanical ventilation (Parikh et al., 16 J. Am. Soc. Nephrol. 3046-52 (2005)).

A marked increase in urinary HGF levels was observed in patients with AKI and was correlated with the disease severity (Taman et al., 48 Clin. Nephrol. 241-45 (1997)). Additionally, HGF values declined to control values in patients recovering from AKI.

A number of recent studies have also demonstrated that the expression of the CXC chemokine interferon-inducible protein-10 (IP-10; CXCL 10) and vascular endothelial growth factor (VEGF) in urine are significantly elevated during kidney allograft rejection (24-26 Matz et al., 69 Kidney Int'l 1683-90 (2006); Peng et al., 35 J. Int'l Med. Res. 442-49 (2007); Tatapudi et al., 65 Kidney Int'l 2390-97 (2004)), and diabetic nephropathy (Kim et al., 67 Kidney Int'l 167-77 (2005); Ruster & Wolf, 13 Front Biosci. 944-55 (2008)).

These nine urinary biomarkers all identified AKI in a cross sectional study of individuals with and without AKI. The diagnostic performance characteristics were best when comparing AKI with healthy individuals; biomarker levels were higher in hospitalized individuals without evidence of AKI than in healthy individuals, accounting for the lower AUC-ROC when including all non-AKI controls. This may relate to the insensitivity and non-specificity of changes in serum creatinine to reflect acute tubular injury. The selection of subjects in the non-disease group in studies of diagnostic performance was important in establishing these base lines. Because urinary biomarkers of AKI is tested in hospitalized patients at risk of AKI, patients admitted to the intensive care unit (ICU) and those undergoing cardiac catheterization (before receiving radiocontrast dye) without clinical evidence of AKI were included in the present study. There is a difference in age of the “healthy volunteers” and cardiac catheterization and ICU controls. The age differential may not explain differences found when comparing AKI patients to non-AKI patients since when studied in animal models: age is associated with increase in urinary markers only if there is an associated age-related incidence of renal disease (Chen et al., 293 Am. J. Physiol. Renal Physiol. F1272-81 (2007)). It is possible that some non-AKI patients in fact had subclinical AKI that was correctly identified by the biomarkers, but were missed when relying on changes in serum creatinine (SCr), leading to an apparent but incorrect reduction in specificity.

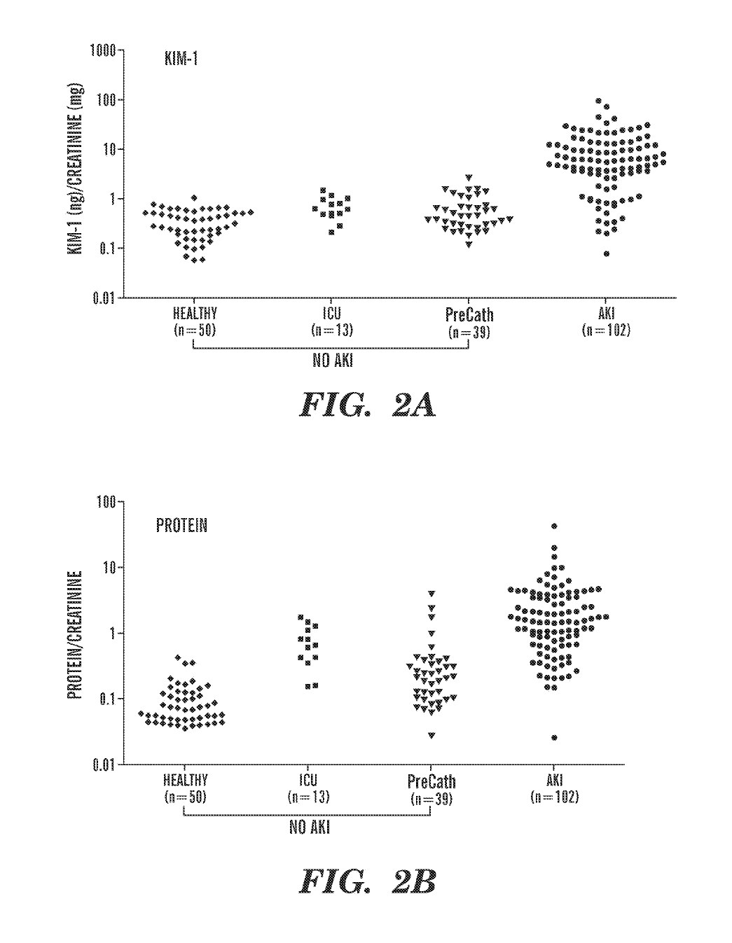

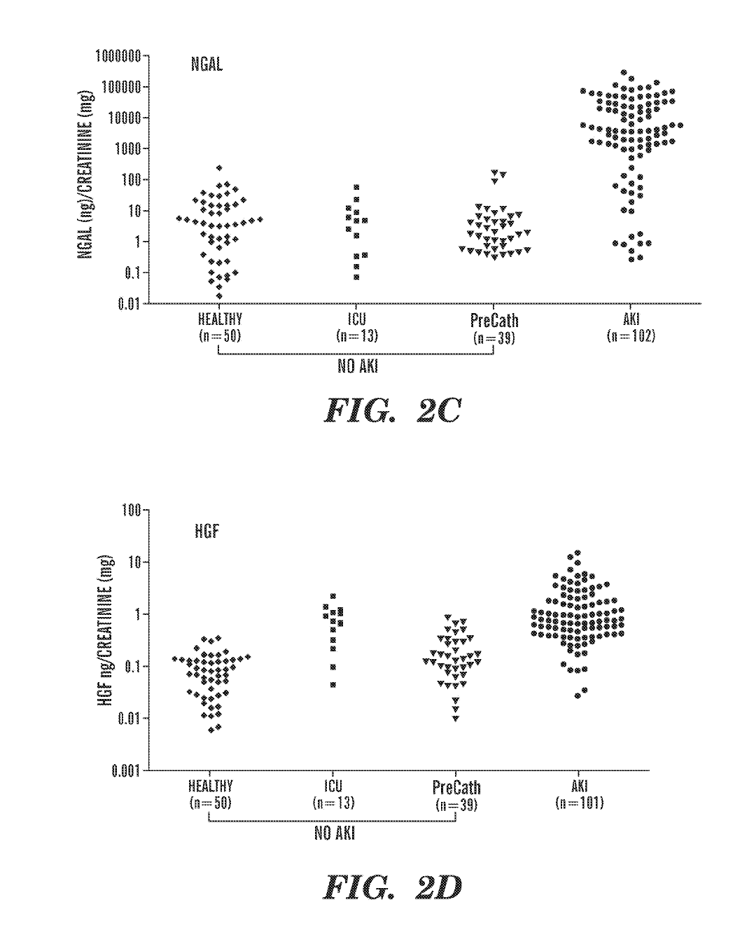

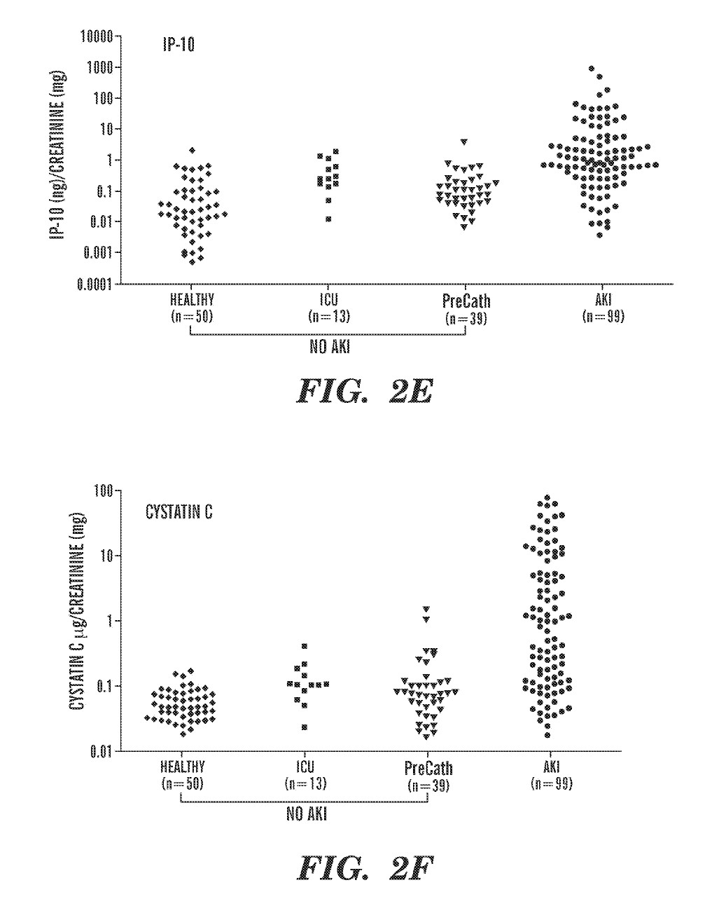

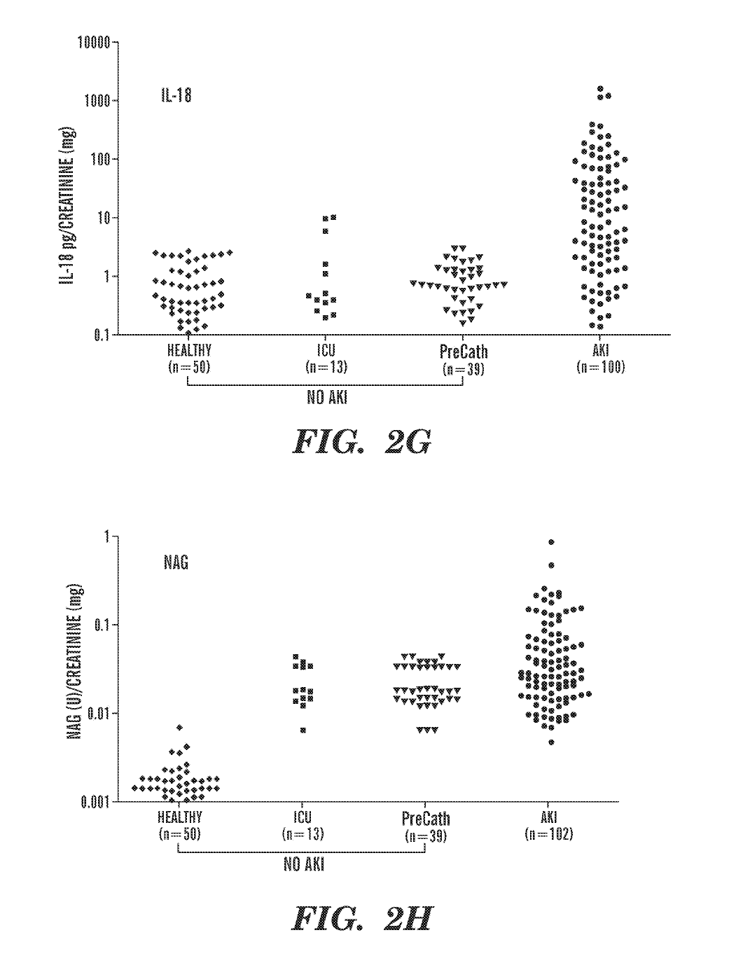

Larger studies comparing long-term outcomes after episodes of AKI may identify other urinary biomarkers for the diagnosis and prognosis of AKI. The non-AKI subjects studied herein also excluded severe chronic kidney disease (CKD) patients, which might also affect the diagnostic performance characteristics of the novel biomarkers. Several urinary biomarkers may be expected to be increased chronically in CKD due to ongoing tubular injury. For an AKI biomarker to retain diagnostic ability in patients with CKD, one would expect levels to increase over baseline after AKI; a cross sectional study may not address that issue, and for that reason subjects with estimated GFR less than 50 ml/min were excluded from the non-AKI control group. The performance of total urinary protein, for example, was excellent in this cross-sectional study. Total urinary protein was higher in patients undergoing cardiac catheterization and ICU controls, however, than in healthy volunteers, and some overlap with AKI patients was evident, raising the possibility of some non-specificity (FIG. 2). Total urinary protein (or perhaps albuminuria) may retain prognostic and diagnostic ability.

Because this study included patients with established AKI at varying stages, it is inappropriate to rank the biomarkers tested according to AUC-ROC. For example, a perfectly sensitive and specific biomarker that increases early after AKI and declines to normal values shortly thereafter might appear to have poor diagnostic ability. Just as troponin, CK-MB, and myoglobin vary in their rate and duration of rise after myocardial infarction, urinary biomarkers may have different kinetics following AKI. The temporal pattern of excretion of urinary biomarkers, important for early diagnosis before rise in SCr may be elucidated. Also, some urine samples were obtained well after the diagnosis of AKI was made, and therefore may not address the issue of early diagnosis prior to a rise in SCr. Prospective studies—in which urine is obtained serially, for example before cardiopulmonary bypass and then at various time points thereafter—are ongoing and assess the temporal pattern of excretion. The biomarkers provided for herein are well suited for such investigations.

Another important role for AKI biomarkers is to provide information about prognosis. Age-adjusted levels of KIM-1, NAG, HGF, total protein, and VEGF predicted death and/or RRT. These findings corroborate reports that KIM-1 and NAG were independent predictors of the composite outcome of death or dialysis in a separate cohort of 201 individuals with established AKI (Liangos et al., 18 J. Am. Soc. Nephrol. 904-12 (2007)). Urinary biomarkers were not compared with generic disease severity scores because of the heterogeneity of the established AKI population in this cohort. There was an inverse correlation between peak SCr and mortality. In other words, patients with higher peak SCr had a lower risk of in-hospital mortality. A paradoxical improvement in outcome with higher SCr was also observed in a previous study of 134 patients with severe AKI requiring RRT (Cerda et al., 22 Nephrol. Dial. Transplant 2781-84 (2007)). Similar findings have been established in the setting of end-stage renal disease and likely relate to the confounding effect of muscle mass and nutritional status (Owen et al., 280 JAMA 1764-68 (1998)).

Microbead technology was used to measure KIM-1 and NGAL, or KIM-1 and HGF, or HGF and IL-18, in the same aliquot of urine sample at the same time. This is important because a single biomarker is rarely adequate to clearly define a particular pathologic state (Fliser et al., 18 J. Am. Soc. Nephrol. 1057-71 (2007); Rifai et al., 24 Nat. Biotech. 971-83 (2006)). An assay that is capable of measuring multiple biomarkers in the same aliquot of biological sample at the same time is extremely useful.

The sensitivity and specificity for diagnosis of AKI was significantly greater by combining the urinary levels of KIM-1, NGAL, HGF and total protein, using the logic regression model of 2.93*(NGAL>5.72 and HGF>0.17)+2.93*(PROTEIN>0.22)−2*(KIM<0.58) than individual biomarkers. The application of logic regression for combination of the multiple biomarkers yielded an AUC of 0.94, exceeding all of the AUC's for the individual biomarkers (for comparison versus all non-AKI controls). Furthermore, the combination of biomarkers confers the advantage of a slightly narrower confidence interval for the AUC, and thus more precise estimation.

Thus, all nine urinary biomarkers performed well in differentiating between patients with and without AKI with AUC-ROCs each greater than 0.83. Using logic regression analysis, the four best performers individually and in combination were KIM-1, NGAL, HGF, and total protein. Confirmation of the utility of this combinatorial approach in prospective studies may be useful in moving kidney injury biomarkers closer to routine clinical use.

The present invention is directed to acute kidney injury biomarkers, and methods and kits comprising the use of agents directed against acute kidney injury biomarkers for facilitating and enhancing the diagnosis of AKI.

### Determining the Levels and Concentrations of Acute Kidney Injury Biomarkers

In one aspect, the invention provides a method for diagnosing acute kidney injury (AKI) in a subject. In one embodiment, the method comprises measuring the concentration of a normalizing protein, such as creatinine, and at least one biomarker in a biological sample obtained from a subject; and comparing the concentration of the at least one biomarker to the concentration of normalizing protein in the sample to determine whether the subject has AKI. In one embodiment, the biological sample is a urine sample. In one embodiment, the at least one biomarker is selected from a panel of biomarkers comprising kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), or chemokine interferon-inducible protein 10 (IP-10; CXCL10). In one embodiment, the at least one biomarker is selected from a panel of biomarkers consisting essentially of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), or chemokine interferon-inducible protein 10 (IP-10; CXCL10). In one embodiment, the at least one biomarker is selected from a panel of biomarkers consisting of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), or chemokine interferon-inducible protein 10 (IP-10; CXCL10). In all such embodiments, a >1.8 fold increase in the concentration of the at least one biomarker over the concentration of the normalizing protein, such as creatinine, indicates that the subject has AKI.

In another embodiment, the method comprises contacting a sample obtained from a subject in need thereof with at least one agent specific for at least one biomarker selected from a panel of biomarkers comprising kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), or chemokine interferon-inducible protein 10 (IP-10; CXCL10), and at least one agent specific for a normalizing protein, such as creatinine, where the agents specific for the least one biomarker and the normalizing protein are used in an assay to determine the level or concentration of the at least one biomarker and the level or concentration of the normalizing protein; and diagnosing a subject with AKI based on the level or concentration of the at least one biomarker present in the sample. In some embodiments, the method further comprises determining a therapeutic treatment for the subject. In one embodiment, the at least one biomarker is selected from a panel of biomarkers consisting essentially of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), or chemokine interferon-inducible protein 10 (IP-10; CXCL10). In one embodiment, the at least one biomarker is selected from a panel of biomarkers consisting of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), or chemokine interferon-inducible protein 10 (IP-10; CXCL10). In one embodiment, the concentration of the at least one biomarker is compared with the concentration of creatinine as the normalizing protein, where a >1.8 fold increase in the at least one biomarker over the creatinine is indicative of AKI in the subject. In other embodiments, the level or concentration of the biomarker protein is measured by measuring the activity of the biomarker.

In one aspect, the method comprises determining the presence or absence of at least two AKI biomarkers (e.g., KIM-1, NGAL, VEGF, Cys, CXCL 10, IL-18, NAG, HGF, or total protein) in a biological sample, e.g., a urine sample, obtained from a patient, wherein the presence of at least one marker is indicative of AKI.

In another embodiment, the methods involve determining the levels or concentrations of at least one AKI biomarker (e.g., KIM-1, NGAL, VEGF, Cys, CXCL 10, IL-18, NAG, HGF, or total protein) in a test sample obtained from a patient being tested for AKI, and comparing the observed levels with the levels of the biomarker found in a control sample, for example a sample obtained from an individual subject or plurality of subjects that do not have AKI. Levels of at least one biomarker higher than levels that are observed in the normal control indicate AKI or risk for AKI. The levels of biomarkers can be represented by arbitrary units, for example as units obtained from a densitometer, luminometer, or an ELISA plate reader.

In one embodiment of the aspect, a secondary diagnostic step can be performed. For example, if a level of at least one AKI biomarker is found to indicate the presence of AKI, then an additional method of detecting the injury can be performed to confirm the injury or further assess the extent of injury. Any of a variety of additional diagnostic steps can be used, such as ultrasound, PET scanning, MRI, or any other imaging techniques, biopsy, clinical examination, ductogram, or any other method.

The present invention further provides for methods of prognostic evaluation of a patient suspected of having, or having, AKI. The method comprises measuring the level of at least one acute kidney injury biomarker, such as an epithelial injury/dediffereniation biomarker (for e.g., KIM-1, NGAL, VEGF, or HGF) present in a test biological sample, for e.g., urine, obtained from a patient and comparing the observed level with a range of at least one AKI biomarker level normally found in biological samples (of the same type) of healthy individuals. A high level for example, corresponds to a poor prognosis, while lower levels indicate that the injury is less severe and corresponds to a better prognosis.

Additionally, resolution of the injury can be assessed by following the levels or concentrations of at least one AKI biomarker in an individual patient. For example, changes in the patients condition can be monitored by comparing changes expression levels of KIM-1, NGAL, VEGF, or HGF in the patient over time. Progressive increases in the levels or concentrations of at least one biomarker is indicative of increased potential for adverse outcome (e.g., mortality). Measuring levels or concentrations of at least one AKI biomarker, as described herein, can be measured by any means known to those skilled in the art. See., e.g., U.S. patent application Ser. No. 11/829,323, including ELISA, multiplex bead, mass spectrometry, and PCR assays. The antibodies for use in the present invention can be obtained from a commercial source, or prepared by well-known methods.

As used herein, the phrase “an increase in the concentration of at least one biomarker over the concentration of normalizing protein” refers to a concentration of at least one biomarker that is greater than a concentration of a normalizing protein present in a biological sample or reference concentration. The terms “increased concentration”, “increase in the level”, “higher level”, or “higher concentration” of a biomarker refers to a level or concentration of a biomarker that is statistically significant or significantly above the level or concentration of that biomarker found in a control or reference sample, in a sample from the same subject at a different timepoint, relative to the level or concentration of a normalizing protein, or relative to a reference concentration or level. As used herein, the phrase “an increase in the concentration of at least one biomarker over the concentration of normalizing protein” refers to a concentration of at least one biomarker that is greater than a concentration of a normalizing protein present in a biological sample. The “higher level” or “increase in the level” can be for example 1.2-fold or higher, for example, 1.8-fold higher or higher, 1.9-fold higher or higher, at least 2-fold higher, or even 3-fold higher. Similarly, an AUC value of about 0.78 may be considered statistically significant. For purposes of comparison, the test sample and control sample are from the same sample type, that is, obtained from the same biological source. The control or reference sample can also be a standard sample that contains the same concentration of the AKI biomarker that is normally found in a biological sample that is obtained from a healthy individual. Alternatively, the control may be a normalizing protein found in the biological sample of the patient that may be used to normalize the AKI biomarkers, such as creatinine. In one embodiment, the term “higher level” or “increase in the level” of the biomarker refers to an increase in the level of at least one biomarker in a sample from a subject, of at least 5% compared to a reference value or a normalizing protein value. In one embodiment it is preferred that an increase in the level of a biomarker is at least 10%, at least 15%, at least 20%, at least 35%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 1-fold, at least 1.2-fold, at least 1.8-fold, at least 1.9-fold, at least 2-fold, at least 3-fold, at least 5-fold, at least 10-fold, at least 25-fold, at least 50-fold, at least 100-fold, at least 1000-fold or more higher than a reference level, for example, the level of the at least one biomarker in a sample from an individual not having acute kidney injury. In another embodiment, a decrease in the level of at least one biomarker, e.g., urinary biomarker, is at least 20%, at least 30%, at least 40%, at least 50%, at least 60%, at least 70%, at least 80%, at least 90%, at least 95%, at least 99%, or even 100% (i.e., absent) compared to a reference level. In an alternate embodiment, the “difference in the normalized level” refers to a statistically significant change (either an increase or decrease) in level of at least one biomarker, e.g., a urinary biomarker, compared to a reference level.

As used herein, the phrase “normalizing the level of the biomarker” or “normalizing the level of the urinary biomarker” refers to the conversion of a data value representing the level of a biomarker (e.g., urinary biomarker, such as KIM-1) in a sample by dividing it by the expression data value representing the level of total protein or a normalizing protein (e.g., creatinine) in the sample, thereby permitting comparison of normalized biomarker values among a plurality of samples, or to one or more reference samples or reference values.

As used herein, the term “normalizing protein” or “normalizing factor” refers to a protein against which the amounts of a biomarker of interest are normalized to, to permit comparison of amounts of the protein of interest in different biological samples. In some embodiments, the normalizing protein is creatinine. In some embodiments, the different biological samples are from different subjects. In other embodiments, the different biological samples are from the same subject, but after different timepoints. Generally, a normalizing protein is constitutively expressed and is not differentially regulated between at least two physiological states or conditions from which samples will be analyzed, e.g., given disease and non-disease states. Thus, for example, a normalizing protein does not vary substantially (i.e., <15%, preferably <10%, <7%, <5%, <4%, <3%, <2%, <1% or less) in the presence and absence of e.g., acute kidney disease. In one embodiment, a normalizing protein is selected based on the degree of correlation (e.g., lowest amount of scatter or lowest standard deviation among replicates) of the protein measured over a series of sample dilutions, compared to the predicted relationship of the dilution series (e.g., predicted by linear regression). In this embodiment, a normalizing protein is selected that has the highest degree of correlation (e.g., as compared to another protein in a protein sample subjected to the same measurement) for measured protein levels assessed over the dilution series. The term “highest degree of correlation” refers to a standard deviation for protein measurements (e.g., replicate measurements) over a dilution series of less than 2 compared to the predicted relationship over the dilution series; preferably the standard deviation is less than 1.5, less than 1, less than 0.5, less than 0.1, less than 0.01, less than 0.001 or more, including a standard deviation of zero (e.g., measured and predicted values are the same). In some embodiments, the normalizing protein is the product of a “housekeeping gene”. As referred to herein, the term “housekeeping gene” refers to a gene encoding a protein that is constitutively expressed, and is necessary for basic maintenance and essential cellular functions. A housekeeping gene generally is not expressed in a cell- or tissue-dependent manner, most often being expressed by all cells in a given organism. Some examples of normalizing proteins encoded by housekeeping genes include e.g., actin, tubulin, GAPDH, among others. In one embodiment, a housekeeping gene product is used as a normalizing protein.

The invention provides, in part, a variety of assay formats that can be used to determine the concentration or level of a biomarker or a normalizing protein. Examples of assay formats include known techniques such as Western blot analysis, radioimmunoassay (hereinafter referred to as “RIA”), Immunoradiometric assay (IRMA), chemiluminescent immunoassays, such as enzyme-linked immunosorbent assay (hereinafter referred to as “ELISA”), multiplex bead assays, a fluorescence antibody method, passive haemagglutination, mass spectrometry (such as MALDI/TOF (time-of-flight), SELDI/TOF), liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, and tandem mass spectrometry HPLC. Some of the immunoassays can be easily automated by the use of appropriate instruments such as the IM x™ (Abbott, Irving, Tex.) for a fluorescent immunoassay and Ciba Coming ACS 180™ (Ciba Corning, Medfield, Mass.) for a chemiluminescent immunoassay.

RIA and ELISA provide the benefit of detection sensitivity, rapidity, accuracy, possible automation of procedures, and the like, for the determination of the concentration or level of an acute kidney injury biomarker (Modern Rheumatology 13: 22-26 (2003)), Ohkuni et al., (International Congress Series 1289: 71-74 (2006)), and Mitchell et al., (Mol Microbiol. 5: 1883-8 (1991)). Radioimmunoassay (Kashyap, M. L. et al., J. Clin. Invest., 60:171-180 (1977)) is a technique in which detection antibody can be used after labeling with a radioactive isotope such as 1251. Antibody arrays or protein chips can also be employed, see for example U.S. Patent Application Nos: 20030013208A1; 20020155493A1; 20030017515 and U.S. Pat. Nos. 6,329,209; 6,365,418, which are herein incorporated by reference in their entirety.

The most common enzyme immunoassay is the “Enzyme-Linked Immunosorbent Assay (ELISA). There are different forms of ELISA which are well known to those skilled in the art, e.g. standard ELISA, competitive ELISA, and sandwich ELISA. The standard techniques for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904. ELISA is a technique for detecting and measuring the concentration of an antigen, such as an acute kidney injury biomarker, using a labeled (e.g. enzyme linked) form of the antibody. In a “sandwich ELISA”, an antibody is linked to a solid phase (i.e. a microtiter plate) and exposed to a biological sample containing antigen (e.g. an acute kidney injury biomarker). The solid phase is then washed to remove unbound antigen. A labeled antibody (e.g. enzyme linked) is then bound to the plate bound-antigen (if present) forming an antibody-antigen-antibody sandwich. Examples of enzymes that can be linked to the antibody are alkaline phosphatase, horseradish peroxidase, luciferase, urease, and B-galactosidase. The enzyme linked antibody reacts with a substrate to generate a colored reaction product that can be measured. In a “competitive ELISA”, a specific concentration of an antibody specific for at least one AKI biomarker is incubated with a sample containing an acute kidney injury biomarker. The acute kidney injury biomarker-antibody mixture is then contacted with a solid phase (e.g. a microtiter plate) that is coated with a acute kidney injury biomarker. The more acute kidney injury biomarker present in the sample, the less free antibody that will be available to bind to the solid phase. A labeled (e.g., enzyme linked) secondary antibody is then added to the solid phase to determine the amount of primary antibody bound to the solid phase.

In some embodiments, the concentration of each of a plurality of biomarkers can be determined simultaneously, in a multiplex fashion, by ELISA (enzyme-linked immunosorbent assay). The sample can be, for example, one of a plurality of samples obtained at one of the various timepoints from a subject in need. In some embodiments, the sample is a human urine sample from a subject, to be tested for determining the concentration of at least one biomarker according to the methods described herein. The sample (e.g., urine) from the individual may further be serially diluted, according to the needs of the assay, and as known to one of ordinary skill in the art. In some embodiments, one or more of a plurality of antibodies or antigen-binding fragments specific for each of the at least one biomarker being assayed in a sample is contacted with the sample to bind any biomarker present in the sample, thus forming a biomarker-antibody complex or biomarker-antigen-binding fragment complex. In some embodiments, each antibody or antigen-binding fragment specific for a biomarker is labeled with a different label. In some embodiments, each different label is a fluorescent label. In all such embodiments, each different label has a unique emission spectra, such that each antibody can be detected individually. The levels or concentrations of each of the biomarkers can then be determined by calculating changes in the emission spectrum, wherein the relative intensity of signal from each of the fluorescent labels correlates with the number of antibodies against the particular biomarker being assayed. For example, a well that displays a more intense signal of the label on the antibody against KIM-1 will have a greater concentration of KIM-1 than a well with a weak signal for that particular label. The wells can be normalized to a well comprising all of the necessary ELISA reagents with the exception of the sample. A series of standards having known concentrations of each of the various biomarkers being assayed permits actual quantification of the concentration of each of the biomarkers in the sample.

In some aspects, the concentration or level of one or more biomarkers can be determined simultaneously, in a multiplex fashion, using a multiplex bead assay. For example, in one embodiment, beads of different sizes or colors (emission spectra) are used for multiplexed immunoassays to determine the concentration of each of a plurality of biomarkers. In some embodiments of this aspect, a plurality of beads of different sizes are coated with different antibodies, wherein each bead of a specific size is conjugated to an antibody specific for a single biomarker. Accordingly, each bead can be differentiated by its unique light scatter characteristics. A sample, such as a urine sample, to be assayed for the presence of at least one biomarker is then contacted with a plurality of beads of different sizes, forming a bead-biomarker conjugate, and the concentrations of each of the at least one biomarker can then be ascertained by, for example, performing flow cytometric analyses on the bead bound-sample.

In some embodiments of this aspect, such bead-based technology can be employed wherein bead populations are identified by one type of fluorescence, while the biomarker-dependent signal is generated by detection reagents carrying a second type of fluorescent signal, thus creating a bead set specific for a plurality of acute kidney injury biomarkers. In preferred embodiments, the distinguishable bead populations are prepared by staining the beads with two or more fluorescent dyes at various ratios. Each bead having a specific ratio of the two or more fluorescent dyes is conjugated to an antibody specific for one of a plurality of biomarkers, thus assigning each bead a unique fluorescent signature. The immunoassay signal is generated by detection reagents, coupled to a third type of fluorescent dye. A sample to be assayed for the presence of at least one biomarker is then contacted with the plurality of beads with unique fluorescent signatures and biomarker specificity, forming a bead-biomarker conjugate for any biomarker present in the sample. The concentrations of each of the at least one biomarker can be ascertained by flow cytometric analyses on the bead bound-sample. For example, in some embodiments, beads are dyed with fluorochromes having different fluorescence intensities. In some embodiments, the beads are 7.5 Am in diameter. In some embodiments, the fluorescent dye incorporated in the beads fluoresces strongly at 650 nm upon excitation with an argon laser. Each bead population of a given fluorescence intensity represents a discrete population for constructing an immunoassay for a single biomarker. Each bead population having a given fluorescence intensity upon excitation is covalently coupled with an antibody directed against a specific biomarker. For example, an antibody directed against KIM-1. These antibody-bound bead populations, each of which are unique in their fluorescence emission intensity, serve as capture beads for a specific biomarker in a sample.

Accordingly, as defined herein a “capture bead” is a bead having a unique fluorescence emission intensity conjugated to an antibody specific for a biomarker. When these capture beads specific for different biomarkers are used as a mixture, the levels of individual biomarkers, such as KIM-1 and HGF, can be simultaneously measured within a given sample. In some embodiments, detection is further mediated by the binding of a specific detection antibody, for example, an antibody that detects any bead-biomarker complex present in a sample, that is directly conjugated with phycoerythrin (PE), to each of the corresponding capture bead-biomarker complexes present in the sample, thus providing a second fluorescent signal for each capture bead. The fluorescent signal is proportional to the concentration of the biomarker in the sample. Separately established calibration curves can be used to determine the concentration of each biomarker in the test sample, using dedicated analysis software, such as CBA software. The data collected using a flow cytometer include information about the physical and spectral parameters of the beads, such as size and the fluorescence emission characteristics of each bead population. These fluorescence emission characteristics include the fluorescent emission of the dyed beads, and the potential fluorescent emissions of the detection fluorochrome (for example, phycoerythrin). When samples are analyzed using a flow cytometer in conjunction with a typical data acquisition and analysis package (for e.g., BD CellQuest™ software), a list-mode data file is saved using a flow cytometry standard file format, FCS. The data stored in the FCS files can be reanalyzed to determine the median fluorescence intensities (MFI) of the various bead populations, defined by their unique physical and spectral characteristics, to then compare reference samples with unknowns. The level of the biomarkers being assayed within individual samples can then be calculated from calibration curves generated by serial dilutions of standard analyte solutions of known concentration. An automated or semiautomated analysis method can be used for rapid reanalysis of the data stored in each FCS file. For example, BD CBA Software is written in the Microsoft® Excel Visual Basic for Applications (VBA) programming language. The CBA Software can recognize FCS 2.0 and 3.0 format data files and automates the identification of CBA bead populations and the determination of detector fluorochrome MFI values for each bead population within the data file for a single sample. Using this data analysis function of the CBA Software for multiple standard files, the MFI values for standards are then determined and plotted. From the plotted standard curve and complex mathematical interpolation, values for unknown samples can be rapidly determined in comparison to known standards using the software.

Other techniques can be used to detect the acute kidney injury biomarkers as required to practice the methods described herein, according to a practitioner's preference, and based upon the present disclosure. The suitability of a given method of distinguishing acute kidney injury biomarkers will depend on the ability of that method or assay to distinguish between the acute kidney injury biomarkers. Thus, an immunoassay can distinguish on the basis of selective binding of one and not another agent. Spectrometric approaches can be applied when a given agent will have a distinct spectrum or profile in the assay relative to others. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Detectably labeled antibodies that specifically bind to the biomarker can then be used to assess acute kidney injury biomarker levels or concentrations, where the intensity of the signal from the detectable label corresponds to the amount of acute kidney injury biomarker present. Levels can be quantitated, for example by densitometry.

In other embodiments, the levels of the various acute kidney injury biomarkers, such as, for example, KIM-1 and HGF, present in a sample can be determined by mass spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference in their entirety.

The terms “mass spectrometry” or “MS” as used herein refer to methods of filtering, detecting, and measuring ions based on their mass-to-charge ratio, or “m/z.” In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). See, e.g., U.S. Pat. No. 6,204,500, entitled “Mass Spectrometry From Surfaces;” U.S. Pat. No. 6,107,623, entitled “Methods and Apparatus for Tandem Mass Spectrometry;” U.S. Pat. No. 6,268,144, entitled “DNA Diagnostics Based On Mass Spectrometry;” U.S. Pat. No. 6,124,137, entitled “Surface-Enhanced Photolabile Attachment And Release For Desorption And Detection Of Analytes;” Wright et al., “Proteinchip surface enhanced laser desorption/ionization (SELDI) mass spectrometry: a novel protein biochip technology for detection of prostate cancer biomarkers in complex protein mixtures,” Prostate Cancer and Prostatic Diseases 2: 264-76 (1999); and Merchant and Weinberger, “Recent advancements in surface-enhanced laser desorption/ionization-time of flight-mass spectrometry,” Electrophoresis 21: 1164-67 (2000), each of which is hereby incorporated by reference in its entirety, including all tables, figures, and claims. Mass spectrometry methods are well known in the art and have been used to quantify and/or identify biomolecules, such as proteins and hormones (see, e.g., Li et al., (2000), Tibtech. 18:151-160; Starcevic et. al., (2003), J. Chromatography B, 792: 197-204; Kushnir MM et. al. (2006), Clin. Chem. 52:120-128; Rowley et al. (2000), Methods 20: 383-397; and Kuster and Mann (1998), Curr. Opin. Structural Biol. 8: 393-400). Further, mass spectrometric techniques have been developed that permit at least partial de novo sequencing of isolated proteins. Chait et al., (1993), Science, 262:89-92; Keough et al., (1999), Proc. Natl. Acad. Sci. USA. 96:7131-6; reviewed in Bergman (2000), EXS 88:133-44. Various methods of ionization are known in the art. For examples, Atmospheric Pressure Chemical Ionisation (APCI) Chemical Ionisation (CI) Electron Impact (EI) Electrospray Ionisation (ESI) Fast Atom Bombardment (FAB) Field Desorption/Field Ionisation (FD/FI) Matrix Assisted Laser Desorption Ionisation (MALDI) and Thermospray Ionisation (TSP) In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. See, e.g., U.S. Pat. No. 5,118,937 (Hillenkamp et al.), and U.S. Pat. No. 5,045,694 (Beavis & Chait). In SELDI, the substrate surface is modified so that it is an active participant in the desorption process. In one variant, the surface is derivatized with adsorbent and/or capture reagents that selectively bind the biomarker of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material. For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094. Detection and quantification of the biomarker will typically depend on the detection of signal intensity. For example, in certain embodiments, the signal strength of peak values from spectra of a first sample and a second sample can be compared (e.g., visually, by computer analysis etc.), to determine the relative amounts of particular biomarker. Software programs such as the Biomarker Wizard program (Ciphergen Biosystems, Inc., Fremont, Calif.) can be used to aid in analyzing mass spectra. The mass spectrometers and their techniques are well known to those of skill in the art. The various assays are described herein in terms of the detection of biomarkers present in the urine. However, its should be understood that the assays can be readily adapted to detect other analytes as needed for various other embodiments and in various other sample types, such as blood or plasma.

The prognostic methods of the invention also are useful for determining a proper course of treatment for a patient having AKI. A course of treatment refers to the therapeutic measures taken for a patient after diagnosis or after treatment for injury.

The present invention is also directed to commercial kits for the detection and prognostic evaluation of AKI. The kit can be in any configuration well known to those skilled in the art and is useful for performing one or more of the methods described herein for the detection of at least one AKI biomarker. The kits are convenient in that they supply many, if not all, of the essential reagents for conducting an assay for the detection of at least one AKI biomarker in a urine test sample, such as described herein. In addition, the assay may be performed simultaneously with a standard or multiple standards included in the kit, such as a predetermined amount of at least one acute kidney injury biomarker (e.g., epithelial injury/dedifferentiation biomarker protein or nucleic acid), so that the results of the test can be quantified or validated.

In one embodiment, the kit comprises a means for detecting levels of at least one AKI biomarker in a sample of urine. The kit may comprise a “dipstick” with at least one AKI biomarker binding agent immobilized thereon, which specifically binds an AKI biomarker protein. Specifically bound AKI biomarker can then be detected using, for example, a second antibody that is detectably labeled with a calorimetric agent or radioisotope.

In other embodiments, the assay kits may contain components for competitive and non-competitive assays, radioimmunoassay (RIA), multiplex bead assays, bioluminescence and chemiluminescence assays, fluorometric assays, sandwich assays, immunoradiometric assays, dot blots, enzyme linked assays including ELISA, microtiter plates, or immunocytochemistry. For each kit the range, sensitivity, precision, reliability, specificity, and reproducibility of the assay are established by means well known to those skilled in the art.

### Methods of Optimizing Treatments for Acute Kidney Injury

Other aspects of the invention provide methods for improving the efficacy of treatment for acute kidney injury, by determining the levels or concentrations of biomarkers.

Accordingly, in one embodiment of this aspect, the method comprises (a) measuring a level or concentration of at least one biomarker in a panel of biomarkers comprising a kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10); and (b) comparing the level or concentration of the at least one biomarker with a reference level or concentration of the at least one biomarker, wherein an increase in the level or concentration of at least one biomarker in a panel of biomarkers comprising KIM-1, NGAL, IL-18, HGF, Cys, NAG, VEGF, and CXCL10 in the sample relative to the reference level or concentration of said at least one biomarker indicates a need to administer to the subject a therapeutic treatment for acute kidney injury. In other embodiments, the panel of biomarkers consists essentially of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10). In other embodiments, the panel of biomarkers consists of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10). In some embodiments, the biological sample is a urine sample.

In another embodiment of this aspect, the method comprises contacting a biological sample obtained from a subject with at least one agent specific for at least one biomarker in a panel of biomarkers comprising a kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10); (b) measuring the level or concentration of the at least one biomarker using an assay specific for the at least one agent; and (c) comparing the level or concentration of the at least one biomarker with a reference level or concentration of the at least one biomarker, wherein an increase in the level or concentration of at least one biomarker in a panel of biomarkers comprising KIM-1, NGAL, IL-18, HGF, Cys, NAG, VEGF, and CXCL10 in the sample relative to the reference level or concentration of said at least one biomarker indicates a need to administer to the subject a therapeutic treatment for acute kidney injury. In other embodiments, the panel of biomarkers consists essentially of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10). In other embodiments, the panel of biomarkers consists of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10),In some embodiments, the biological sample is a urine sample.

In another embodiment of this aspect, a method for monitoring treatment efficacy of a subject with acute kidney injury is provided, the method comprising: (a) determining, from a biological sample obtained from a subject at a first time point, a level or concentration of at least one biomarker in a panel of biomarkers comprising kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10); (b) determining a level or concentration of said at least one biomarker in a panel of biomarkers from a sample obtained from said subject at a second time point; and (c) comparing the level or concentration of the at least one biomarker in a panel of biomarkers at the second time point to the level or concentration of the at least one biomarker in a panel of biomarkers at the first time point, wherein a decrease in the level or concentration of the at least one biomarker at said second time point indicates the treatment is efficacious for said subject, and wherein an increase in the level or concentration of the at least one biomarker at said second time point indicates the treatment is not efficacious for said subject. In other embodiments, the panel of biomarkers consists essentially of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10). In other embodiments, the panel of biomarkers consists of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10). In some embodiments, the biological sample is a urine sample.

The management of acute kidney injury hinges, in part, on identification and treatment of the underlying cause. In addition to treatment of the underlying disorder, management of acute kidney injury can include the avoidance of substances that are toxic to the kidneys, or “nephrotoxins,” which include, but are not limited to, non-steroidal anti inflammatory drugs (NSAIDs), such as ibuprofen, iodinated contrasts, such as those used for CT scans, and others.

The choice of a specific therapeutic treatment for acute kidney injury is dependent, in part, on the cause of the acute renal injury, i.e., whether the cause of the acute kidney injury is pre-renal, renal instrinsic, or post-renal. For example, in pre-renal acute kidney injury in the absence of fluid overload, administration of intravenous fluids is typically the first step to improve renal function. Fluid administration may be monitored, for example, with the use of a central venous catheter to avoid over- or under-replacement of fluid. In situations where low blood pressure is a persistent problem in the fluid replete patient, inotropes, such as norepinephrine and dobutamine, may be given to improve cardiac output and hence renal perfusion. In some embodiments, dopamine may be administered. In cases of prerenal acute kidney injury induced by toxins, discontinuation of the offending agent, such as aminoglycoside, penicillin, NSAIDs, or acetaminophen, can be an effective treatment. If the cause of acute kidney injury is obstruction of the urinary tract, relief of the obstruction (with a nephrostomy or urinary catheter) may be necessary.

In cases where the acute kidney injury has renal intrinsic causes, specific therapies and treatment regimens are administered based on the nature of the renal intrinsic cause. For example, intrinsic acute kidney injury due to Wegener's granulomatosis may respond to steroid medication.

Renal replacement therapy, such as hemodialysis or continuous venovenous hemofiltration (CVVH), may be instituted in some cases of acute kidney injury. Metabolic acidosis and hyperkalemia, the two most serious biochemical manifestations of acute renal failure, may require medical treatment with sodium bicarbonate administration and antihyperkalemic measures, unless dialysis is required.

In some cases of acute kidney injury, lack of improvement after treatment with fluid resuscitation, therapy-resistant hyperkalemia, metabolic acidosis, or fluid overload may necessitate artificial support in the form of dialysis or hemofiltration.

In some cases of acute kidney injury, in which end-stage renal failure has occurred, treatment involves a kidney transplant. As defined herein, a “kidney transplant” or “renal transplant” is the organ transplant of a kidney into a patient with end-stage renal disease. Kidney transplantation is typically classified as deceased-donor (formerly known as cadaveric) or living-donor transplantation depending on the source of the recipient organ. Living-donor renal transplants are further characterized as genetically related (living-related) or non-related (living-unrelated) transplants, depending on whether a biological relationship exists between the donor and recipient.

The efficacy of a given treatment for acute kidney injury can be determined by the skilled clinician, for example, using the criteria discussed herein. However, a treatment is considered “effective treatment,” as the term is used herein, if any one or all of the signs or symptoms of acute kidney injury, such as in one example, urine creatinine levels, are altered in a beneficial manner, other clinically accepted symptoms or markers of disease are improved, or even ameliorated, e.g., by at least 10% following treatment. Efficacy can also be measured by a failure of an individual to worsen as assessed by hospitalization or need for medical interventions (i.e., progression of the disease is halted or at least slowed). Methods of measuring these indicators are known to those of skill in the art and/or are described herein. Treatment includes any treatment of a acute kidney injury disease in an individual or an animal (some non-limiting examples include a human, or a mammal) and includes: (1) inhibiting the disease, e.g., arresting, or slowing the progression of acute kidney injury or acute kidney injury complications; or (2) relieving the disease, e.g., causing regression of symptoms, e.g., normalizing or reducing urine creatinine levels; and (3) preventing or reducing the likelihood of the development of a further acute kidney injury complication, or the need for administration of a further treatment, such as for example, a renal transplant.

An effective amount for the treatment of a disease means that amount which, when administered to a mammal in need thereof, is sufficient to result in effective treatment as that term is defined herein, for that disease.

### Systems for Determining Acute Kidney Injury Biomarker Levels and Concentrations

Other aspects of the invention also provide for systems (and computer readable media for causing computer systems) to perform a method for determining the expression value of an acute kidney injury biomarker (e.g., urinary biomarker).

In some aspects, embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including any suitable combination of the foregoing.

Computer-readable data embodied on one or more computer-readable media may define instructions, for example, as part of one or more programs, that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments of the invention include at minimum a determination system #40, a storage device #30, a comparison module #80, and a display module #110. The functional modules can be executed on one, or multiple, computers, or by using one, or multiple, computer networks. The determination system has computer executable instructions to provide e.g., fluorescence information in computer readable form.

The determination system #40, can comprise any system for detecting a signal from one or more protein binding agents, e.g., a fluorescently labeled antibody that binds an acute kidney injury biomarker. Such systems can include flow cytometry systems, fluorescence assisted cell sorting systems, fluorescence microscopy systems (e.g., fluorescence microscopy, confocal microscopy), any ELISA detection system and/or any Western blotting detection system.

The information determined in the determination system can be read by the storage device #30. As used herein the “storage device” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage devices also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device is adapted or configured for having recorded thereon expression level or protein level information. Such information may be provided in digital form that can be transmitted and read electronically, e.g., via the Internet, on diskette, via USB (universal serial bus) or via any other suitable mode of communication.

As used herein, “stored” refers to a process for encoding information on the storage device. Those skilled in the art can readily adopt any of the presently known methods for recording information on known media to generate manufactures comprising expression level information.

In one embodiment, the reference data stored in the storage device to be read by the comparison module is chromogenic data or fluorescence emission data obtained from an ELISA or a multiplex bead determination system #40.

The “comparison module” #80 can use a variety of available software programs and formats for the comparison operative to compare fluorescence data determined in the determination system to reference samples and/or stored reference data. In one embodiment, the comparison module is configured to use pattern recognition techniques to compare information from one or more entries to one or more reference data patterns. The comparison module may be configured using existing commercially-available or freely-available software for comparing patterns, and may be optimized for particular data comparisons that are conducted. The comparison module provides computer readable information related to normalized expression level of a acute kidney injury biomarker, the chronic kidney injury status of an individual, efficacy of treatment in an individual, and/or method for treating an individual.

The comparison module, or any other module of the invention, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

The comparison module provides a computer readable comparison result that can be processed in computer readable form by predefined criteria, or criteria defined by a user, to provide a content based in part on the comparison result that may be stored and output as requested by a user using a display module #110.

The content based on the comparison result, may be a normalized expression value compared to a reference that shows whether an individual has a chronic kidney disease.

In one embodiment of the invention, the content based on the comparison result is displayed on a computer monitor #120. In one embodiment of the invention, the content based on the comparison result is displayed through printable media #130, #140. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces.

The present invention therefore provides for systems (and computer readable media for causing computer systems) to perform methods for assessing whether an individual has an acute kidney injury.

Systems and computer readable media described herein are merely illustrative embodiments of the invention for performing methods of assessing whether an individual has a chronic kidney injury, and are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

The present invention can further be defined in any of the following numbered paragraphs:

1. A method for diagnosing acute kidney injury (AKI) in a subject comprising the steps of: measuring a concentration or level of a normalizing protein and measuring a concentration or level of at least one of the following biomarkers: kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), or chemokine interferon-inducible protein 10 (IP-10; CXCL10) in a biological sample obtained from a subject; and comparing the concentration or level of said biomarker with the concentration or level of the normalizing protein, wherein a >1.8 fold increase in the concentration or level of at least one biomarker over the concentration or level of normalizing protein is indicative that the subject has AKI.
2. The method of paragraph 1, wherein the concentration or level of the at least one biomarker protein is detected using an antibody-based binding agent which specifically binds to the biomarker protein.
3. The method of paragraph 1, wherein the concentration or level of the biomarker protein is measured by measuring an activity of the biomarker.
4. The method of paragraph 1, wherein the normalizing protein is creatinine.
5. The method of paragraph 1, wherein the biological sample is a urine sample.
6. A method for determining whether a subject has a kidney infection or a bladder infection, the method comprising measuring a level or concentration of kidney injury molecule-1 (KIM-1) protein in a biological sample obtained from a subject, and comparing it to a reference level or concentration of KIM-1, wherein a reference level or concentration of KIM-1 in the biological sample is indicative of bladder infection, and wherein a higher level of KIM-1 in the biological sample obtained from the subject as compared with a reference level is indicative of kidney infection.
7. The method of paragraph 6, wherein the biological sample is a urine sample.
8. A method for diagnosing acute kidney injury (AKI) in a subject in need thereof comprising the steps of: (i) measuring a level or concentration of a normalizing protein in a biological sample obtained from a subject in need thereof; (ii) measuring a level or concentration of at least one of the following biomarkers: kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), or chemokine interferon-inducible protein 10 (IP-10; CXCL10) in the biological sample; wherein one or more agents are exposed to said biological sample prior to at least one of the steps of said measuring of the level or concentration of the normalizing protein and said measuring of the level or concentration of said at least one biomarker; and (iii) comparing the level or concentration of said at least one biomarker with the level or concentration of the normalizing protein, wherein a >1.8 fold increase in the level or concentration of at least one biomarker over the level or concentration of normalizing protein is indicative of AKI.
9. A method for diagnosing acute kidney injury in a subject in need thereof, the method comprising: (i) contacting a biological sample obtained from a subject in need thereof with at least one detectable agent specific for at least one of the following biomarkers: kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), or chemokine interferon-inducible protein 10 (IP-10; CXCL10); wherein one or more agents are exposed to said biological sample prior to at least one of the step of said measuring of the level or concentration of normalizing protein and said measuring of the level or concentration of said at least one biomarker; and (ii) comparing the level or concentration of said at least one biomarker with the level or concentration of normalizing protein, wherein a >1.8 fold increase in the level or concentration of at least one biomarker over the level or concentration of normalizing protein is indicative of AKI.
10. The method of paragraphs 8 or 9, wherein the biological sample is a urine sample.
11. The method of paragraphs 8 or 9, wherein the normalizing protein is creatinine.
12. A computer readable storage medium having computer readable instructions recorded thereon to define software modules for implementing on a computer a method for assessing a biomarker level or concentration in a biological sample, said computer readable storage medium comprising:

(a) instructions for storing and accessing data representing a level or concentration of a biomarker and a level or concentration of a normalizing protein for a biological sample obtained from a subject in need thereof;

(b) instructions for normalizing said level or concentration of said biomarker to said level or concentration of said normalizing protein via a normalization module, thereby producing a normalized level or concentration of said biomarker,

(c) instructions for displaying retrieved content to a user, wherein the retrieved content comprises a normalized biomarker level or concentration.

13. The computer readable storage medium of paragraph 12, further comprising instructions for comparing said normalized level or concentration of said biomarker to reference data stored on said storage device using a comparison module, whereby a change in the biomarker level or concentration is determined.
14. The method of paragraph 13, wherein the normalizing protein is creatinine.
15. A computer system for obtaining data from a biological sample obtained from at least one subject, the system comprising:

(a) a specimen container to hold a biological sample;

(b) a determination module configured to determine read-out information, wherein said read-out information comprises
1) information representing a level or concentration of a normalizing protein, and
2) information representing a level or concentration of at least one biomarker measured in the biological sample,

(c) a storage device configured to store data output from said determination module,

(d) a normalization module configured to normalize information representing a level or concentration of said at least one biomarker to information representing a level or concentration of said normalizing protein;

(e) a display module for displaying retrieved content to the user, wherein the retrieved content comprises a normalized biomarker level.

16. The computer system of paragraph 15, further comprising a comparison module adapted to compare the data obtained from said normalization module with reference data on said storage device, whereby a change in the level or concentration of said biomarker is determined.
17. The method of paragraph 15, wherein the normalizing protein is creatinine.
18. A method for diagnosing acute kidney injury (AKI) in a subject comprising the steps of: obtaining a urine sample from said subject; calculating the area under the curve-receiver operating characteristics (AUC-ROC) for at least one of the following biomarkers: kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), or chemokine interferon-inducible protein 10 (IP-10; CXCL10); normalized to urinary creatinine; wherein an AUC-ROC of >0.78 for at least one of said biomarkers is indicative of AKI.
19. A method for diagnosing acute kidney injury in a human subject, the method comprising: (a) measuring a level or concentration of at least one biomarker in a panel of biomarkers comprising kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10) in a biological sample obtained from a subject; and (b) comparing said level or concentration of said at least one biomarker with a reference level or concentration of said at least one biomarker, wherein an increase in the level or concentration of at least one biomarker in a panel of biomarkers comprising KIM-1, NGAL, IL-18, HGF, Cys, NAG, VEGF, and CXCL10 in the sample relative to the reference level or concentration of said at least one biomarker is indicative of the diagnosis of acute kidney injury in the human subject.
20. A method for diagnosing acute kidney injury in a human subject, the method comprising: (a) measuring a level or concentration of at least one biomarker in a panel of biomarkers consisting essentially of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10) in a biological sample obtained from a subject; and (b) comparing said level or concentration of said at least one biomarker with a reference level or concentration of said at least one biomarker, wherein an increase in the level or concentration of at least one biomarker in a panel of biomarkers consisting essentially of KIM-1, NGAL, IL-18, HGF, Cys, NAG, VEGF, and CXCL10 relative to the reference level or concentration of said at least one biomarker is indicative of the diagnosis of acute kidney injury in the human subject. 21. A method for diagnosing acute kidney injury in a human subject, the method comprising: (a) measuring a level or concentration of at least one biomarker in a panel of biomarkers consisting of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10) in a biological sample obtained from a subject; and (b) comparing said level or concentration of said at least one biomarker with a reference level or concentration of said at least one biomarker, wherein an increase in the level or concentration of at least one biomarker in a panel of biomarkers consisting of KIM-1, NGAL, IL-18, HGF, Cys, NAG, VEGF, and CXCL10 relative to the reference level or concentration of said at least one biomarker is indicative of the diagnosis of acute kidney injury in the human subject.
22. The method of any of paragraphs 19, 20, or 21, wherein the biological sample is a urine sample.
23. A method for diagnosing acute kidney injury in a human subject, the method comprising: (a) contacting a biological sample obtained from a subject with an agent specific for at least one biomarker in a panel of biomarkers comprising kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10), thus forming at least one agent-biomarker complex; (b) determining a level or concentration of the at least one biomarker in the biological sample by performing an assay specific for the at least one agent-biomarker complex; (c) comparing the level or concentration of the at least one biomarker in the biological sample with a reference level or concentration of at least one biomarker, wherein an increase in the level or concentration of at least one biomarker in the panel of biomarkers comprising KIM-1, NGAL, IL-18, HGF, Cys, NAG, VEGF, and CXCL10 relative to the reference level or concentration of at least one biomarker is indicative of the diagnosis of acute kidney injury in the subject.
24. A method for diagnosing acute kidney injury in a human subject, the method comprising: (a) contacting a biological sample obtained from a subject with an agent specific for at least one biomarker in a panel of biomarkers consisting essentially of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10), thus forming at least one agent-biomarker complex; (b) determining a level or concentration of the at least one biomarker in the biological sample by performing an assay specific for the at least one agent-biomarker complex; (c) comparing the level or concentration of the at least one biomarker in the biological sample with a reference level or concentration of at least one biomarker, wherein an increase in the level or concentration of at least one biomarker in the panel of biomarkers consisting essentially of KIM-1, NGAL, IL-18, HGF, Cys, NAG, VEGF, and CXCL10 relative to the reference level or concentration of at least one biomarker is indicative of the diagnosis of acute kidney injury in the subject.
25. A method for diagnosing acute kidney injury in a human subject, the method comprising: (a) contacting a biological sample obtained from a subject with an agent specific for at least one biomarker in a panel of biomarkers consisting of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10), thus forming at least one agent-biomarker complex; (b) determining a level or concentration of the at least one biomarker in the biological sample by performing an assay specific for the at least one agent-biomarker complex; (c) comparing the level or concentration of the at least one biomarker in the biological sample with a reference level or concentration of at least one biomarker, wherein an increase in the level or concentration of at least one biomarker in the panel of biomarkers consisting of KIM-1, NGAL, IL-18, HGF, Cys, NAG, VEGF, and CXCL10 relative to the reference level or concentration of at least one biomarker is indicative of the diagnosis of acute kidney injury in the subject.
26. The method of any of paragraphs 23, 24, or 25, wherein the biological sample is a urine sample.
27. A computer system for obtaining gene expression data for biomarkers in a biological specimen comprising: (a) a determination system configured to receive level or concentration information from a biological sample obtained from a subject, wherein the level or concentration information comprises a level or concentration of at least one biomarker in a panel of biomarkers comprising kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10); (b) a storage device configured to store data output from the determination system; (c) a comparison module adapted to compare the data stored on the storage device with reference and/or control data, and to provide a retrieved content, and (d) a display module for displaying a page of the retrieved content for the user, wherein the retrieved content indicates that said subject has acute kidney injury or is at risk for acute kidney injury if there is an increase in the level or concentration of at least one biomarker in the panel of biomarkers comprising KIM-1, NGAL, IL-18, HGF, Cys, NAG, VEGF, and CXCL10 relative to the reference and/or control data.
28. A computer system for obtaining gene expression data for biomarkers in a biological specimen comprising: (a) a determination system configured to receive level or concentration information from a biological sample obtained from a subject, wherein the level or concentration information comprises a level or concentration of at least one biomarker in a panel of biomarkers consisting essentially of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10); (b) a storage device configured to store data output from the determination system; (c) a comparison module adapted to compare the data stored on the storage device with reference and/or control data, and to provide a retrieved content, and (d) a display module for displaying a page of the retrieved content for the user, wherein the retrieved content indicates that said subject has acute kidney injury or is at risk for acute kidney injury if there is an increase in the level or concentration of at least one biomarker in the panel of biomarkers consisting essentially of KIM-1, NGAL, IL-18, HGF, Cys, NAG, VEGF, and CXCL10 relative to the reference and/or control data.
29. A computer system for obtaining gene expression data for biomarkers in a biological specimen comprising: (a) a determination system configured to receive level or concentration information from a biological sample obtained from a subject, wherein the level or concentration information comprises a level or concentration of at least one biomarker in a panel of biomarkers consisting of kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10); (b) a storage device configured to store data output from the determination system; (c) a comparison module adapted to compare the data stored on the storage device with reference and/or control data, and to provide a retrieved content, and (d) a display module for displaying a page of the retrieved content for the user, wherein the retrieved content indicates that said subject has acute kidney injury or is at risk for acute kidney injury if there is an increase in the level or concentration of at least one biomarker in the panel of biomarkers consisting of KIM-1, NGAL, IL-18, HGF, Cys, NAG, VEGF, and CXCL10 relative to the reference and/or control data.
30. The method of any of paragraphs 27, 28, or 29, wherein the biological sample is a urine sample.
31. A method for monitoring treatment efficacy of a subject with acute kidney injury, the method comprising: (a) determining, from a biological sample obtained from a subject at a first time point, a level or concentration of at least one biomarker in a panel of biomarkers comprising kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10); (b) determining a level or concentration of said at least one biomarker in a panel of biomarkers from a sample obtained from said subject at a second time point; and (c) comparing the level or concentration of the at least one biomarker in a panel of biomarkers at the second time point to the level or concentration of the at least one biomarker in a panel of biomarkers at the first time point, wherein a decrease in the level or concentration of the at least one biomarker at said second time point indicates the treatment is efficacious for said subject, and wherein an increase in the level or concentration of the at least one biomarker at said second time point indicates the treatment is not efficacious for said subject.
32. A method for improving the efficacy of treatment for acute kidney injury, the method comprising (a) measuring a level or concentration of at least one biomarker in a panel of biomarkers comprising a kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10); and (b) comparing the level or concentration of the at least one biomarker with a reference level or concentration of the at least one biomarker, wherein an increase in the level or concentration of at least one biomarker in a panel of biomarkers comprising KIM-1, NGAL, IL-18, HGF, Cys, NAG, VEGF, and CXCL10 in the sample relative to the reference level or concentration of said at least one biomarker indicates a need to administer to the subject a therapeutic treatment for acute kidney injury.
33. A method for improving the efficacy of treatment for acute kidney injury, the method comprising contacting a biological sample obtained from a subject with at least one agent specific for at least one biomarker in a panel of biomarkers comprising a kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), and chemokine interferon-inducible protein 10 (IP-10; CXCL10), thus forming at least one biomarker-agent complex; (b) measuring a level or concentration of the at least one biomarker using an assay specific for the at least one biomarker-agent complex; and (c) comparing the level or concentration of the at least one biomarker with a reference level or concentration of the at least one biomarker, wherein an increase in the level or concentration of at least one biomarker in a panel of biomarkers comprising KIM-1, NGAL, IL-18, HGF, Cys, NAG, VEGF, and CXCL10 in the sample relative to the reference level or concentration of said at least one biomarker indicates a need to administer to the subject a therapeutic treatment for acute kidney injury.
34. The method of any of paragraphs 31, 32, or 33, wherein the biological sample is a urine sample.

The present invention is further illustrated by the following Examples. These Examples are provided to aid in the understanding of the invention and are not construed as a limitation thereof.

### EXAMPLES

### Example 1

### Selection of Participants

Patients with documented AKI of at least the “Risk” category of the RIFLE criterion (Bellomo et al., 227 J. Immunol. Meths. 41-52 (1999)) (peak SCr >50% increase over admission value or known baseline) were recruited from the inpatient nephrology consultation service. Causes of AKI were obtained by detailed chart review including the treating nephrologist's consultation note and evaluation of laboratory data by a co-author not involved in the patients' care (SSW). Individuals without AKI were selected from three distinct populations: healthy volunteers, patients undergoing cardiac catheterization, and patients admitted to the intensive care unit. Healthy volunteers were excluded if they reported a recent hospitalization, diagnosis of chronic kidney disease, or treatment with nephrotoxic medications (non-steroidal anti-inflammatory drugs were allowed). Patients undergoing cardiac catheterization and those admitted to the intensive care unit were included in the non-AKI cohort if they had normal urine output (>0.5 ml/kg/hr), stable SCr during hospitalization (<0.3 mg/dL change from baseline), and an estimated GFR >50 ml/min. Urine samples from cardiac catheterization patients were taken before administration of intravenous contrast. All participants were patients or employees (healthy volunteers) of Brigham and Women's Hospital, a tertiary care teaching hospital. The Institutional Review Board approved the protocols for recruitment and sample collection.

Urine test samples were collected from spontaneous voids or from indwelling Foley catheters. Urine dipstick analysis was performed (Multistix 8 SG, Bayer Corp.), followed by centrifugation and microscopic examination of the urine sediment (Olympus microscope). The urine supernatant was aliquoted into 1.8 ml eppendorf tubes and frozen within 2 hours of collection at −80° C. At the time of assay samples were thawed, vortexed, and centrifuged at 14,000 rpm at 4° C. and 30 μl-100 μl of supernatant was pipetted for biomarker measurement. Assays were performed within three months of urine collection after a maximum of three freeze-thaw cycles. Urine samples from patients with established AKI were collected close to the time of initial consultation.

Urinary total protein (Sigma) and NAG (Roche diagnostics) were measured spectrophotometrically according to the manufacturers' protocols. Urinary Cystatin C was measured as reported previously (8) with the N latex Cystatin C kit (Dade Behring, Marburg, Germany) using a BN II nephelometer. KIM-1, NGAL, IL-18, HGF, IP-10, VEGF were measured using micro-bead based assays described below.

### Example 2

### Development and Evaluation of Micro-Bead Based Assay for Urinary Biomarker Quantitation

Coupling of the beads to respective capture antibodies: The microbead based assays for KIM-1 and NGAL were developed and evaluated in this study using an amine coupling Kit from Bio-Rad whereas microbead assays for HGF, IL-18, VEGF, and IP-10 were commercially available from Bio-Rad laboratories.

Evaluation of the assay: The performance characteristics of the microbead based assay was evaluated in the same way as the Kim-1 ELISA (Vaidya et al., 290 Am. J. Physiol. Renal. Physiol. F517-29 (2006)), by measuring the sensitivity, assay range, specificity, reproducibility, recovery, and interference (Table 1, below). The sensitivity or the lowest limit of detection (LLD) was determined by diluting the respective standard in sample diluent; the concentration which is two standard deviations above the background “sample diluent alone” was determined to be the LLD. The analytical recovery in control and diseased urines was determined by adding a known amount (low, medium and high concentrations) of respective recombinant proteins into urine of control/healthy volunteers or diseased urine samples and quantitating the levels of respective antigens prior to and For subsequent to the addition. This was done to verify that there were no interfering substances in the urine of patients with AKI (Oda et al., 48 Clin. Chem. 1445-53 (2002)). Dilutional linearity was evaluated in normal and diseased urines to justify sample dilution, which was needed for all the assays to eliminate the interference in antigen recovery for KIM-1, NGAL, IL-18, HGF, VEGF, and IP10 assays. Sample dilution was required for NAG, total protein and cystatin C assays in order to fit the concentrations of respective antigens in the linear range of the standard curve. Diseased urine samples containing low, medium, and high concentrations of respective antigens (as measured by the microbead based assay) were diluted 1:2, 1:10, 1:20, 1:100, 1:500 using sample diluent.

Statistics. Continuous variables were expressed as means±SD or medians, and compared using the student's t-test or Kruskal-Wallis test, as appropriate. Categorical variables were expressed as proportions and compared with the 2-test. Urinary creatinine concentration was used to normalize biomarker measurements in order to account for the influence of urinary dilution on biomarker concentrations. Scatterplots were used to graphically display log-transformed normalized biomarker levels in the four groups of subjects. Diagnostic performance (i.e., the ability of a urinary biomarker to identify AKI) was assessed by evaluating sensitivity and specificity using the receiver operating characteristics (ROC) curve. The area under the ROC curve (AUC) and 95% confidence interval (CI) were calculated using the non-parametric method (Hanley & McNeil, 148 Radiology 839-43 (1983)). The AUC for a diagnostic test ranges from 0.5 (no better than chance alone) to 1.0 (perfect test, equivalent to the gold standard).

Logic regression (Ruczinski et al., 12 J. Computational & Graphical Stats. 475-511 (2003)), was used to construct Boolean combinations of binary coded biomarkers to allow for high-order interactions between the biomarker outcomes. To apply this methodology, indicator variables for the biomarkers were created using their 33rd and 67th quantiles from the non-AKI subjects. Cross-validation was used to select the optimal number of logic trees and total leaves (i.e., complexity) in the model. Free software for this approach was obtained from the Fred Hutchinson Cancer Research Center in Seattle, Department of Biostatistics. The resulting score was then used to construct an ROC curve. The bootstrap percentile method (2000 replications) was used to obtain a 95% confidence interval for the AUC. A similar approach was taken by Janes et al., (24 Stat. Med. 1321-38 (2005)), in an analysis of screening for colorectal cancer. The ability of urinary biomarkers to predict in-hospital mortality and identify the need for renal replacement therapy in patients with established AKI was tested using logistic regression analysis, adjusting for age. Two-tailed P value of <0.05 was considered statistically significant.

### Example 3

### Urinary Biomarkers in Individuals with and without Acute Kidney Injury Quantitation of Biomarkers

The microbead based assays for KIM-1 and NGAL were developed and evaluated in this study whereas all other biomarker assays were commercially available. The sensitivity, specificity, precision profile, recovery, interference and dilutional linearity for each assay were extensively evaluated and were within the acceptable range (Table 1).

TABLE 1
Evaluation of assays to measure biomarkers for acute kidney injury.

| | | | | | | | Cystatin | | |
|---|---|---|---|---|---|---|---|---|---|
| Parameters | KIM-1 | NGAL | IL-18 | HGF | VEGF | 1P-10 | C | NAG | Protein |
| Assay | MBS | MBS | MBS | MBS | MBS | MBS | LBBT | ESBC | ASBC |
| Principle | ELISA | ELISA | ELISA | ELISA | ELISA | ELISA | | | |
| LLD | 4.4 pg/ml | 0.53 ng/ml | 0.125 pg/ml | 0.709 pg/ml | 10 pg/ml | 32 pg/ml | 0.043 mg/L | 0.2 U/L | 0.011 |
| Assay | 40-160000 | 0.49-1000 | 0.12-2000 | 0.7-1446 | 7.8-31,982 | 25-10,000 | 0.043-27.2 | 0.2-52.9 | 0.01-2 |
| Range | pg/ml | ng/ml | pg/ml | pg/ml | pg/ml | pg/ml | mg/l | U/L | mg/ml |
| Intra assay | <15% | <15% | <10% | <10% | <10% | <10% | <5% | <2% | <10% |
| Inter assay | <20% | <20% | <20% | <20% | <20% | <20% | <5% | <2% | <10% |
| Recovery | 85%-100% | 85%-100% | 85%-110% | 85%-110% | 85%-110% | 85%-110% | 90%-100% | 90%-100% | 90%-100% |
| Linearity | 1:2, | 1:10, | 1:10, | 1:2, | 1:5, | 1:20, | 1:20, | 1:2, | 1:2, |
| (linear over | 1:10, | 1:100, | 1:20, | 1:10, | 1:10, | 1:100, | 1:100, | 1:10, | 1:5 |
| dilutions) | 1:20 | 1:500 | 1:40 | 1:20 | 1:20 | 1:400 | 1:400 | 1:20 | |

| | |
|---|---|
| Interference | No interference when tested for albumin, bilirubin, creatinine, glucose, hemoglobin, urea. |
| | Unknown interference does exists with the human KIM-1 and NGAL assay, but dilution |
| | of the sample with diluent results in 85%-100% recovery. |

MBS ELISA: Microbead based sandwich ELISA; LBBT: Latex bead based turbidimetry; ESBC: Enzyme-substrate based colorimety; ASBC: Absorbance shift based colorimetry.

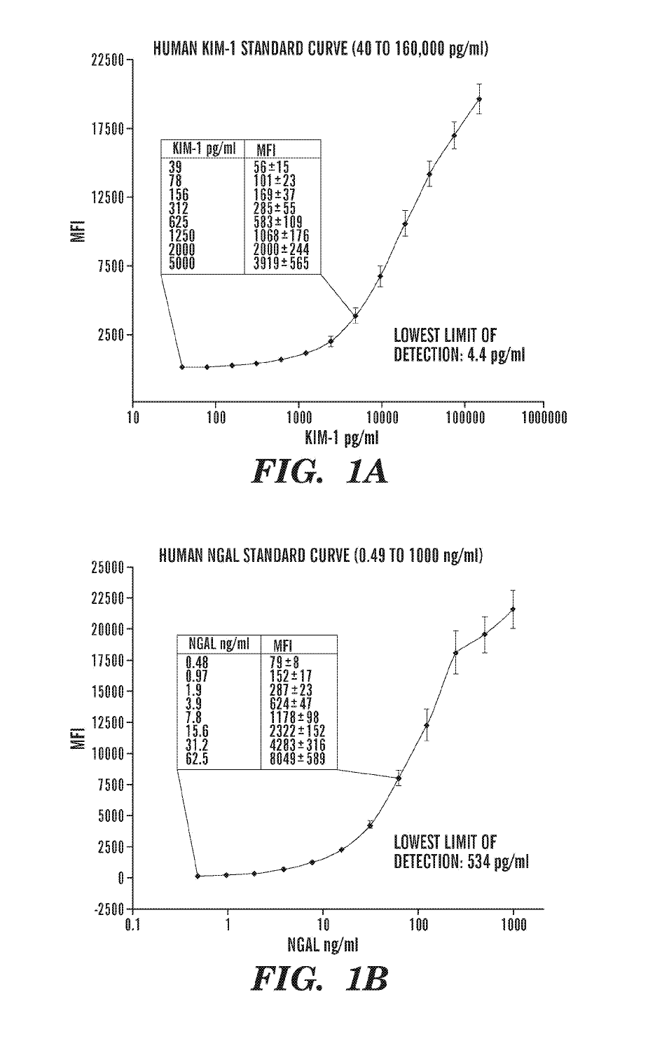

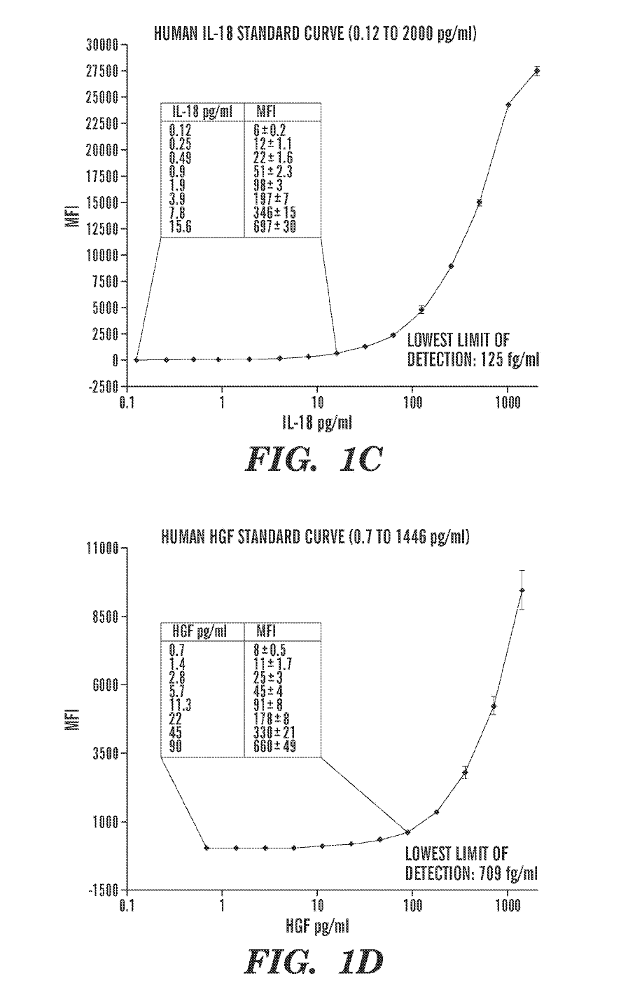

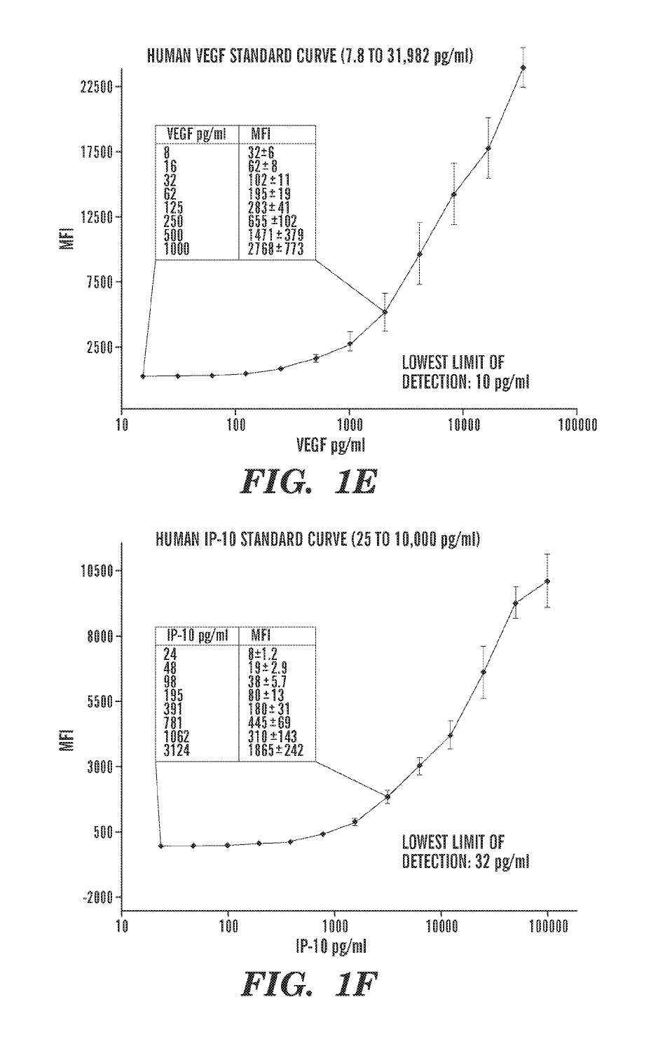

Urinary biomarker values were calculated using a 12 to 14.5 parametric logarithmic standard curve (FIG. 1). Human subjects: Urinary biomarkers were measured in 102 patients with established AKI from a variety of causes and in 102 individuals without AKI as follows: 39 patients undergoing cardiac catheterization, 13 patients admitted to the intensive care unit, and 50 healthy volunteers. Demographic and clinical information are shown in Table 2.

Urinary biomarker values were calculated using a 12 to 14.5 parametric logarithmic standard curve (FIG. 1). Human subjects: Urinary biomarkers were measured in 102 patients with established AKI from a variety of causes and in 102 individuals without AKI as follows: 39 patients undergoing cardiac catheterization, 13 patients admitted to the intensive care unit, and 50 healthy volunteers. Demographic and clinical information are shown in Table 2.

TABLE 2
Demographic and clinical characteristics of human subjects

| | Established acute | Cardiac | Intensive | Healthy |
|---|---|---|---|---|
| | kidney injury | catheterization | care unit | volunteers |
| | (N = 102) | (N = 39) | (N = 13) | (N = 50) |
| Mean age, | 61.2 ± 17.2 | 69.1 ± 14.1 | 67.7 ± 13.2 | 35.7 ± 10.6 |
| years, ± SD* | | | | |
| Female** | 45% | 36% | 31% | 76% |
| Black^{§} | 11% | 10% | 0% | 10% |
| Cause of AKI or | Sepsis (34%), ischemia | — | Postoperative | — |
| reason for ICU | (18%), nephrotoxin exposure | | complications | |
| admission^{†} | (15%), post-cardiac surgery | | (54%), trauma | |
| | (13%), radiocontrast admin- | | (32%), sepsis | |
| | istration (11%), pre-renal | | (14%) | |
| | azotemia (10%), other (25%) | | | |
| Serum creatinine | Peak: range 1.7- | Median 1.0 mg/dL | Median 0.7 mg/dL | —^{‡} |
| | 10.0 mg/dL | Range 0.6 to 1.4 | Range 0.4 to 1.0 | |
| | Required renal | mg/dL | mg/dL | |
| | replacement therapy:46% | | | |

*Statistically significant pairwise comparisons between AKI vs cardiac catheterization (P = 0.01), AKI vs healthy volunteers (P < 0.001), and AKI vs non-AKI (P = 0.001).
**P < 0.001
^{§}P = 0.75
^{†}Sum exceeds 100% in AKI due to multiple diagnoses in individual patients
^{‡}Healthy volunteers were excluded if they reported a diagnosis of chronic kidney disease; serum creatinine was not measured

Diagnostic ability of urinary biomarkers: Median urinary concentrations of cystatin C, HGF, IL-18, IP-10, KIM-1, NAG, NGAL, total protein, and VEGF were each significantly higher in patients with AKI than in those without AKI (P<0.001). A scatterplot of the distribution of biomarkers levels is shown in FIG. 2. Each of the nine urinary biomarkers was able to differentiate between the established AKI and non-AKI groups (P<0.001). The diagnostic performances were best when defining the non-AKI group as healthy volunteers, but remained high for most biomarkers when comparing AKI versus all non-AKI (i.e., including cardiac catheterization and intensive care unit patients without AKI). NAG had nearly perfect diagnostic ability (AUC-ROC 1.00) when comparing AKI to healthy individuals, but had substantially lower diagnostic performance when all non-AKI individuals (AUC-ROC 0.83) were included. The same phenomenon was observed for VEGF (AUC-ROC 0.90 versus 0.73). By contrast, the diagnostic performance characteristics of cystatin C, HGF, IL-18, IP-10, KIM-1, NGAL, and total protein were comparable (i.e., overlapping 95% CI for AUC-ROC) irrespective of the non-AKI groups with which the AKI group was compared (Table 3).

TABLE 3
Comparative diagnostic performance characteristics of urinary biomarkers for the identification
of established AKI using the area under the receiver operating characteristics curve (AUC-ROC).

| | AKI (N = 102) vs | | | | AKI (N = 102) vs all | | | |
|---|---|---|---|---|---|---|---|---|
| | healthy individuals (N = 50) | | | | non-AKI controls (N = 102) | | | |
| | AUC-ROC | | | | AUC-ROC | | | |
| Biomarker* | (95% CI) | Cutoff | Sensitivity | Specificity | (95% CI) | Cutoff | Sensitivity | Specificity |
| Urine creatinine (mg) | 0.78 | 62 | 67% | 76% | 0.72 | 37 | 45% | 92% |
| | (0.70-0.84) | | | | (0.65-0.78) | | | |
| Cystatin C (ug/mg) | 0.90 | 0.11 | 78% | 94% | 0.85 | 0.12 | 78% | 83% |
| | (0.84-0.94) | | | | (0.80-0.90) | | | |
| HGF (ng/mg) | 0.96 | 0.23 | 91% | 94% | 0.89 | 0.37 | 84% | 84% |
| | (0.92-0.99) | | | | (0.84-0.93) | | | |
| IL-18 (pg/mg) | 0.85 | 2.30 | 69% | 92% | 0.83 | 2.74 | 68% | 95% |
| | (0.78-0.90) | | | | (0.77-0.88) | | | |
| IP-10 (ng/mg) | 0.89 | 0.13 | 85% | 80% | 0.84 | 0.62 | 69% | 89% |
| | (0.83-0.93) | | | | (0.79-0.89) | | | |
| KIM-1 (ng/mg) | 0.95 | 0.70 | 90% | 96% | 0.93 | 1.73 | 80% | 99% |
| | (0.90-0.98) | | | | (0.88-0.96) | | | |
| NAG (U/mg) | 1.00 | 0.007 | 99% | 100% | 0.83 | 0.015 | 80% | 65% |
| | (0.98-1.00) | | | | (0.77-0.88) | | | |
| NGAL (ng/mg) | 0.89 | 83.0 | 80% | 98% | 0.89 | 82.7 | 80% | 96% |
| | (0.83-0.94) | | | | (0.84-0.93) | | | |
| Protein (mg/mg) | 0.98 | 0.22 | 96% | 94% | 0.91 | 0.46 | 81% | 87% |
| | (0.94-1.00) | | | | (0.87-0.95) | | | |
| VEGF (ng/mg) | 0.90 | 0.43 | 77% | 84% | 0.73 | 0.64 | 62% | 62% |
| | (0.84-0.94) | | | | (0.66-0.79) | | | |

Cross-validation of logic regression models that included up to three trees with up to seven total leaves resulted in a model with three trees and four leaves as optimal. This model corresponds to a risk score of 2.93*(NGAL>5.72 and HGF>0.17)+2.93*(PROTEIN>0.22)−2*(KIM<0.58) and was derived comparing AKI versus all non-AKI combined. The ROC curve that evaluated sensitivity and specificity was constructed for every threshold value for this derived risk score. The AUC for this combination of biomarkers is 0.94 (95% bootstrap percentile confidence interval (CI): 0.901, 0.969).

Prognostic ability of urinary biomarkers: Individuals with established AKI had an in-hospital mortality rate of 36%; 46% of these patients required renal replacement therapy (RRT); and 60% had the composite outcome of death or RRT. Table 4 shows median biomarker values in patients with established AKI according to clinical outcome.

TABLE 4
Median normalized biomarker levels in patients with
established AKI, according to clinical outcome

| | In hospital | | | Renal replacement | | | Mortality or renal | | |
|---|---|---|---|---|---|---|---|---|---|
| | mortality (36%) | | | therapy (46%) | | | replacement therapy (60%) | | |
| | | | P | | | P | | | P |
| | Died | Survived | value | Yes | No | value | Yes | No | value |
| Cystatin C | 1.19 | 0.72 | 0.63 | 1.21 | 0.69 | 0.87 | 1.03 | 0.85 | 0.60 |
| (ug/mg) | | | | | | | | | |
| HGF | 1.23 | 0.77 | 0.07 | 1.13 | 0.76 | 0.24 | 1.15 | 0.74 | 0.03 |
| (ng/mg) | | | | | | | | | |
| IL-18 | 16.89 | 6.12 | 0.27 | 16.22 | 5.90 | 0.29 | 15.19 | 4.93 | 0.29 |
| (pg/mg) | | | | | | | | | |
| IP-10 | 1.21 | 0.97 | 0.74 | 1.25 | 0.92 | 0.66 | 1.38 | 0.85 | 0.29 |
| (ng/mg) | | | | | | | | | |
| KIM-1 | 10.17 | 5.19 | 0.008 | 7.24 | 5.19 | 0.37 | 6.84 | 4.80 | 0.10 |
| (ng/mg) | | | | | | | | | |
| Protein | 2.20 | 1.51 | 0.13 | 2.21 | 1.14 | 0.02 | 2.20 | 1.13 | 0.02 |
| (mg/mg) | | | | | | | | | |
| NGAL | 5384.4 | 3113.2 | 0.94 | 12883.3 | 2063.0 | 0.14 | 6389.1 | 2044.3 | 0.40 |
| (ng/mg) | | | | | | | | | |
| NAG | 0.05 | 0.03 | 0.02 | 0.06 | 0.02 | 0.003 | 0.06 | 0.02 | <0.001 |
| (U/mg) | | | | | | | | | |
| VEGF | 1.63 | 0.91 | 0.07 | 1.24 | 0.95 | 0.11 | 1.55 | 0.75 | 0.008 |
| (ng/mg) | | | | | | | | | |

All biomarker values normalized to urinary creatinine. P value represents results from logistic regression analysis using log-transformed biomarker levels, adjusting for age.

In age-adjusted analyses using log-transformed biomarker values, the following were significant predictors of outcome: HGF composite of death/RRT); KIM-1 (mortality); total protein (RRT and composite mortality/RRT); NAG (mortality, RRT, and composite of mortality/RRT); and VEGF (composite of mortality/RRT). Peak SCr was associated inversely with mortality (age-adjusted odds ratio, 0.78, 95% CI 0.62-0.99) but not with RRT or the composite of mortality/RRT. SCr at the time of sample collection was not significantly associated with mortality and/or RRT.

Urinary biomarkers in AKI of different causes: Urinary biomarkers were compared across diagnostic categories of AKI (Table 5), after establishing for each patient a single most likely diagnosis based on chart review (ATN (including post-cardiac surgery, ischemia, and pigment nephropathy), N=33; sepsis, N=32; contrast nephropathy, N=6; nephrotoxin administration, N=6; and other, N=24). Statistically significant differences in at least one diagnostic category compared to others were observed for HGF (P=0.03), KIM-1 (P<0.007), and NAG (P<0.007); generally, higher levels were seen in ATN and sepsis than in the other causes of AKI.

TABLE 5
Median values (10^{th} and 90^{th} percentiles) of normalized biomarkers in patients
with established AKI, according to most likely single diagnosis.

| | ATN | Sepsis | Contrast | Nephrotoxin | Other* | P |
|---|---|---|---|---|---|---|
| | (N = 33) | (N = 32) | (N = 6) | (N = 6) | (N = 24) | value** |
| Cystatin C | 0.36 | 1.31 | 0.38 | 5.87 | 0.69 | 0.51 |
| (ug/mg) | (0.05-18.71) | (0.06-44.01) | (0.03-15.59) | (0.38-66.38) | (0.08-14.83) | |
| HGF | 1.05 | 1.26 | 0.93 | 0.70 | 0.48 | 0.03 |
| (ng/mg) | (0.21-4.31) | (0.06-44.01) | (0.64-2.25) | (0.21-12.53) | (0.28-1.50) | |
| IL-18 | 5.90 | 32.40 | 2.06 | 15.82 | 6.34 | 0.20 |
| (pg/mg) | (0.61-169.58) | (0.39-283.56) | (0.05-160.80) | (0.30-141.55) | (0.39-103.84) | |
| IP-10 | 2.21 | 1.16 | 0.71 | 1.39 | 0.97 | 0.33 |
| (ng/mg) | (0.39-42.24) | (0.08-54.67) | (0.01-5.56) | (0.01-186.90) | (0.03-26.13) | |
| KIM-1 | 9.90 | 6.78 | 4.53 | 3.92 | 3.45 | 0.007 |
| (ng/mg) | (3.08-30.56) | (0.36-28.10) | (0.01-5.56) | (0.45-25.58) | (0.52-12.53) | |
| NAG | 0.04 | 0.06 | 0.02 | 0.05 | 0.02 | 0.007 |
| (U/mg) | (0.01-0.19) | (0.02-0.22) | (0.01-0.15) | (0.01-0.22) | (0.01-0.04) | |
| NGAL | 5346.0 | 18005.7 | 1486.0 | 1714.0 | 1756.7 | 0.06 |
| (ng/mg) | (0.5-64834.5) | (98.6-97036.6) | (69.0-74134.3) | (972.3-172795.0) | (22.9-30325.1) | |
| Protein | 1.60 | 1.82 | 1.47 | 1.16 | 1.54 | 0.50 |
| (mg/mg) | (0.36-10.09) | (0.59-6.75) | (0.52-3.28) | (0.04-5.30) | (0.22-4.61) | |
| VEGF | 1.23 | 1.74 | 0.64 | 0.63 | 0.67 | 0.15 |
| (ng/mg) | (0.29-55.12) | (0.33-11.88) | (0.10-1.56) | (0.26-61.42) | (0.24-8.59) | |

*Other diagnoses included: pre-renal azotemia (N = 8), acute interstitial nephritis (N = 4), acute on chronic kidney disease without single precipitant (N = 3), acute glomerulonephritis (N = 2), myeloma (N = 2), tumor lysis syndrome (N = 1), urate nephropathy (N = 1), scleroderma renal crisis (N = 1), obstructive uropathy (N = 1), veno-occlusive disease (N = 1)
**Kruskall-Wallis test

### Example 4

### KIM-1 as a Biomarker for Kidney Infection

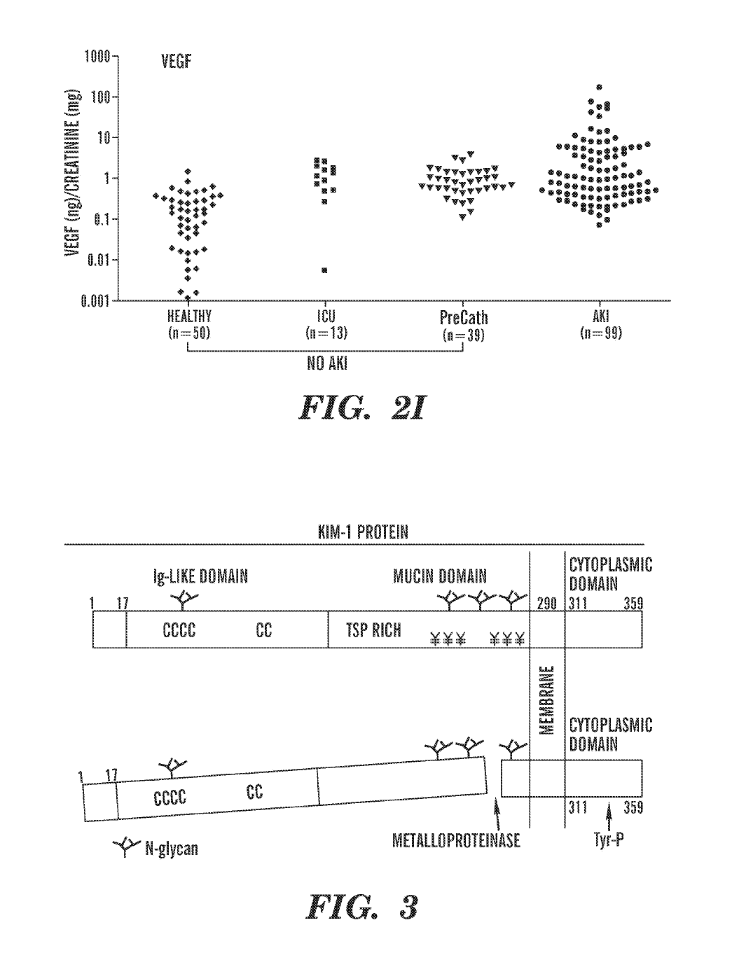

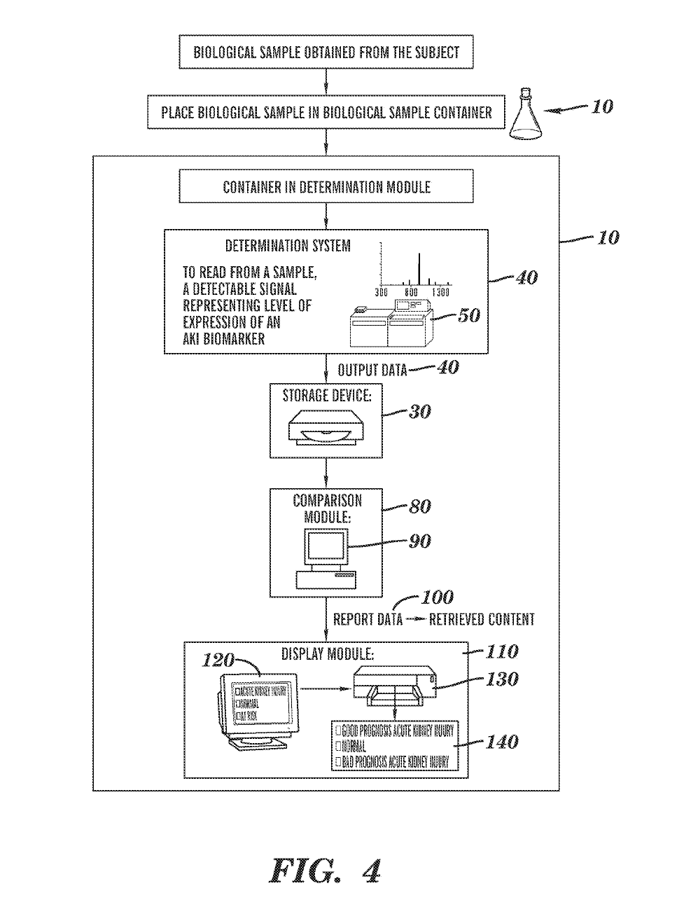

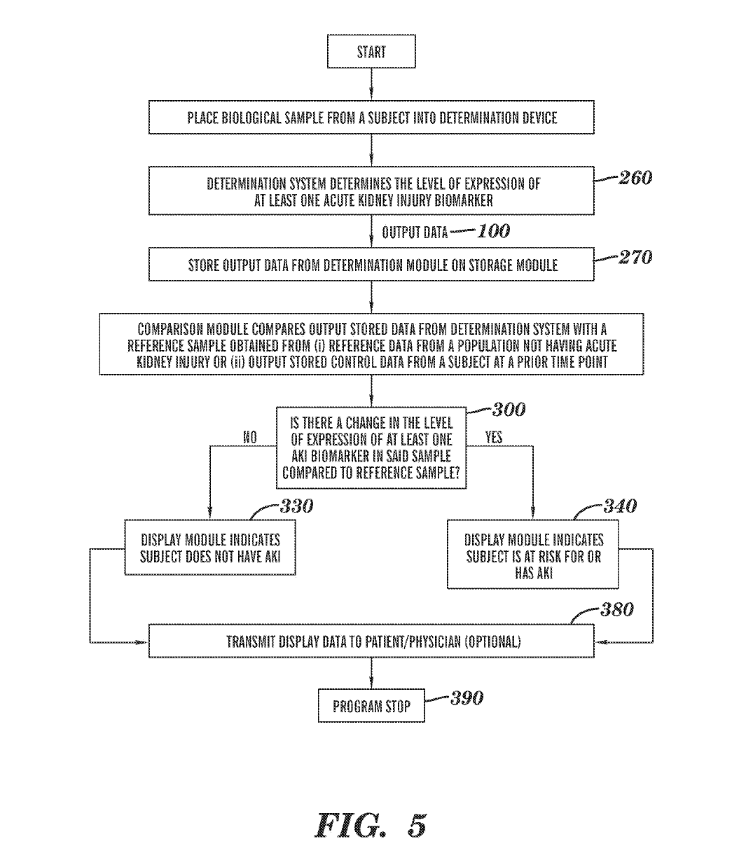

In many animal models and human studies, including the statistical work provided for herein, the expression of KIM-1 is an early and prominent marker of kidney injury or disease. See Bonventre, 68(5241) 78-83 (2008). Kim-1 is a type-1 transmembrane protein with glycosylated mucin and IgG-like domains in the ectodomain of the protein and a relatively short intracellular domain that is tyrosine phosphorylated. The ectodomain is cleaved by metalloproteinases. The intracellular domain has a tyrosine phosphorylation site that may be critical for the regulation of KIM-1 function. A schematic of Kim-1 is shown in FIG. 3.

KIM-1 is produced and shed into the urine following proximal tubular kidney injury, and is not produced in the bladder. Hence, KIM-1 (and or its ectodomain) is increased in the urine of some patients with bladder infections because the infection has reached the upper urinary tract. In a patient with cystitis alone, not suffering from pyelonephritis, the level of KIM-1 in a urine test sample is not elevated. Conversely, the urine sample from a cystitis patient found to have an elevated level of KIM-1 (and/or its ectodomain) as compared with control values, indicates that the patient may have pyelonephritis and needs a different clinical intervention. The use of this biomarker facilitates the diagnosis and treatment of pyelonephritis in cystitis patients.

1. A method for diagnosing acute kidney injury (AKI) in a subject comprising the steps of: measuring a level or concentration of a normalizing protein and measuring a level or concentration of at least one of the following biomarkers: kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), or chemokine interferon-inducible protein 10 (IP-10; CXCL10) in a biological sample obtained from a subject; and comparing the level or concentration of said biomarker with the level or concentration of a normalizing protein, wherein a >1.8 fold increase in the level or concentration of at least one biomarker over the level or concentration of normalizing protein is indicative that the subject has AKI.

2. The method of claim 1, wherein the concentration of the at least one biomarker protein is detected using an antibody-based binding agent which specifically binds to the biomarker protein.

3. The method of claim 1, wherein the level or concentration of the biomarker protein is measured by measuring an activity of the biomarker.

4. The method of claim 1, wherein the normalizing protein is creatinine.

5. The method of claim 1, wherein the biological sample is a urine sample.

6. A method for determining whether a subject has a kidney infection or a bladder infection, the method comprising measuring a level or concentration of kidney injury molecule-1 (KIM-1) protein in a biological sample obtained from a subject, and comparing it to a reference level or concentration of KIM-1, wherein a reference level or concentration of KIM-1 in the biological sample obtained from the subject is indicative of bladder infection, and wherein an increase in the level or concentration of KIM-1 in the biological sample obtained from the subject as compared with the reference level or concentration is indicative of kidney infection.

7. The method of claim 6, wherein the biological sample is a urine sample.

8. A method for diagnosing acute kidney injury (AKI) in a subject in need thereof comprising the steps of: (i) measuring a level or concentration of a normalizing protein in a biological sample obtained from a subject in need thereof; (ii) measuring a level or concentration of at least one of the following biomarkers: kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), or chemokine interferon-inducible protein 10 (IP-10; CXCL10) in the biological sample; wherein one or more agents are exposed to said biological sample prior to at least one of the step of said measuring of the level or concentration of the normalizing protein and said measuring of the level or concentration of said at least one biomarker; and (iii) comparing the level or concentration of said at least one biomarker with the level or concentration of the normalizing protein, wherein a >1.8 fold increase in the level or concentration of at least one biomarker over the level or concentration of normalizing protein is indicative of AKI.

9. A method for diagnosing acute kidney injury in a subject in need thereof, the method comprising: (i) contacting a biological sample obtained from a subject in need thereof with at least one detectable agent specific for at least one of the following biomarkers: kidney injury molecule-1 (KIM-1), neutrophil gelatinase associated lipocalin (NGAL), interleukin-18 (IL-18), hepatocyte growth factor (HGF), cystatin C (Cys), N-acetyl-β-D-glucosaminidase (NAG), vascular endothelial growth factor (VEGF), or chemokine interferon-inducible protein 10 (IP-10; CXCL10); wherein one or more agents are exposed to said biological sample prior to at least one of the step of said measuring of a level or concentration of normalizing protein and measuring a level or concentration of said at least one biomarker; and (ii) comparing the level or concentration of said at least one biomarker with the level or concentration of normalizing protein, wherein a >1.8 fold increase in the level or concentration of at least one biomarker over the level or concentration of normalizing protein is indicative of AKI.

10. The method of claim 8, wherein the biological sample is a urine sample.

11. The method of claim 8, wherein the normalizing protein is creatinine.

12-24. (canceled)

25. The method of claim 9, wherein the biological sample is a urine sample.

26. The method of claim 9, wherein the normalizing protein is creatinine.
